(19) United States
(12) Patent Application Publication
(10) Pub. No.: US 2016/0115196 A1
(43) Pub. Date: Apr. 28, 2016

(54) SELF-ASSEMBLED MICRO-AND NANOSTRUCTURES

(71) Applicants: NORTHWESTERN UNIVERSITY, Evanston, IL (US); RAMOT AT TEL-AVIV UNIVERSITY LTD., Tel Aviv (IL)

(72) Inventors: Galit FICHMAN, Yavne (IL); Lihi ADLER-ABRAMOVICH, Herzeliya (IL); Ehud GAZIT, Ramat Hasharon (IL); Phillip B. MESSERSMITH, Clarendon Hills, IL (US)

(73) Assignee: RAMOT AT TEL-AVIV UNIVERSITY LTD., Tel Aviv (IL)

(21) Appl. No.: 14/894,043

(22) PCT Filed: May 28, 2014
(86) PCT No.: PCT/IL2014/050479

§ 371 Date: Nov. 25, 2015

Related U.S. Application Data
(60) Provisional application No. 61/827,785, filed on May 28, 2013.

(51) Int. Cl. C07K 5/065; C07K 7/06; A01N 37/46; C07K 5/087

(52) U.S. Cl. CPC C07K 5/06078; C07K 5/0812; C07K 7/06; A01N 37/46; A61K 38/00

(57) ABSTRACT

The present invention discloses self-assembled bioadhesive anti-microbial, anti-fouling and/or anti-oxidant micro- and nano-structures comprising a plurality of amino acids or peptides, wherein each amino acid is an aromatic amino acid comprising a catecholic moiety, and/or each peptide comprises at least one aromatic amino acid comprising a catecholic moiety. Further disclosed are methods and kits for preparing these micro- and nano-structures. Further disclosed are uses of these micro- and nano-structures in pharmaceutical, cosmetic and medical devices applications.

### FIELD OF THE INVENTION

The present invention relates to self-assembled bioadhesive, anti-microbial, anti-fouling and/or anti-oxidant micro- and nano-structures comprising a plurality of amino acids or peptides, the micro- or nano-structures comprising at least one aromatic amino acid comprising a catecholic moiety. The present invention further relates to methods of preparing the self-assembled micro- and nano-structures and to their use in a variety of biomedical and industrial applications, for example in pharmaceutical and cosmetic compositions and in medical devices.

### BACKGROUND OF THE INVENTION

Bioadhesives are natural polymeric materials with adhesive properties. Bioadhesives may be comprised of a variety of substances, but proteins and carbohydrates feature prominently. Several types of bioadhesives offer adhesion in wet environments and under water, while others can stick to low surface energy—non-polar surfaces, such as plastic. However, the most prominent use of bioadhesives due to the biocompatibility thereof is in biomedical applications, for example in the preparation of biological glues. Current biological glues (fibrin, albumin, gelatin-resorcinol-formaldehyde, etc.) suffer from low bond strength and are in some cases derived from blood products, with associated risk of viral or prion contamination. On the other hand, synthetic glues (e.g., cyanoacrylate adhesives) are very strong but they are also toxic to living tissues and form rigid, nonporous films that can hinder wound healing [1].

Natural adhesion of mussels, other bivalves and algae to rocks and other substrates has been described, for example, in U.S. Pat. No. 5,015,677, U.S. Pat. No. 5,520,727 and U.S. Pat. No. 5,574,134. The adhesion of marine mussels has drawn a particular interest due to their remarkable ability to bind strongly to virtually all inorganic and organic surfaces in wet environment under the conditions in which most adhesives function poorly [2]. In recent studies it was found that every type of mussel adhesive protein (MAP) contains 5-30% of the non-coded amino acid 3,4-dihydroxyphenyl-L-alanine (DOPA), which is obtained by hydroxylation of tyrosine residues. Moreover, the DOPA content in the protein was found to correlate with the protein adhesive strength, such that MAPs, which exhibit strong adhesion and are typically found close to the adhesion interface, comprise a higher proportion of DOPA residues [3]. The exact role of DOPA in MAPs is not fully understood. Nevertheless, the catechol functionality of DOPA residues is thought to be responsible for both cross-linking and adhesion of the MAPs, as it can form several chemical interactions including hydrogen bonding, metal-ligand complexation, Michael-type addition, π-π interactions and quinhydrone charge-transfer complexation [4].

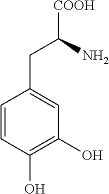 [SMILES: N[C@@H](Cc1ccc(O)c(O)c1)C(=O)O]

### 3,4-dihydroxyphenyl-L-alanine (DOPA)

EP Patent No. 1589088 is directed to biodegradable compositions comprising adhesive, biocompatible polymers and methods used to cover surfaces and to attach structures to eye tissues, such as the cornea. Polyphenolic proteins isolated from mussels (MAPs) are used in conjunction with polysaccharides and pharmaceutically acceptable fine filaments, to achieve strong adhesive bonding.

PCT Patent Application No. WO 2006/038866 discloses an improved coating for biomedical surfaces including a bioadhesive polyphenolic protein derived from a byssus-forming mussel, e.g. Mefp-1 (Mytilus edulis foot protein-1).

Extraction of MAPs from mussels is, however, not practical for commercial scale production. Attempts to mimic mussels' adhesion properties were made, typically using synthetic or genetically engineered polypeptides containing amino acid motifs derived from mussel adhesives, or by incorporating DOPA into synthetic polymers.

U.S. Pat. No. 4,908,404 discloses a water soluble cationic peptide-containing graft copolymer exhibiting a number average molecular weight of from about 30,000 to about 500,000 comprising: (a) a polymeric backbone containing or capable of modification to include free primary or secondary amine functional groups for reaction with an amino acid or peptide graft and exhibiting a number average molecular weight from about 10,000 to about 250,000; and (b) an amino acid or peptide graft reacted with from at least about 5% to about 100% of the primary or secondary amine functional groups of the polymeric backbone, wherein said amino acid or peptide graft comprises at least one 3,4-dihydroxyphenylalaine (DOPA) amino acid or a precursor thereof capable of hydroxylation to the DOPA form.

U.S. Patent Application No. 2005/0201974 is directed to polymers with improved bioadhesive properties and to methods for improving bioadhesion of polymers, wherein a compound containing an aromatic group which contains one or more hydroxyl groups is grafted onto a polymer or coupled to individual monomers, and wherein, in a preferred embodiment, the polymer is a polyanhydride and the aromatic compound is the catechol derivative, DOPA.

EP Patent Application No. 0242656 discloses methods for forming bioadhesive polyphenolic proteins containing 3,4-dihydroxyphenylalanine residues from protein precursors containing tyrosine residues.

U.S. Patent Application No. 2003/0087338 to one of the inventors of the present invention is directed to a route for the conjugation of DOPA moieties to various polymeric systems, including poly(ethylene glycol) or poly(alkylene oxide) systems such as poly(ethylene oxide)-poly(propylene oxide)-poly(ethylene oxide) (PEO-PPO-PEO) block copolymers.

Additional polymers that are end functionalized with DOPA groups are described in scientific literature [5-8]. Furthermore, Statz et. al. describe DOPA-modified oligomeric peptides that were developed to generate stable antifouling surface coatings [9].

A key element in future nanotechnology is the use of nanostructures fabricated through molecular self-assembly [10]. In a bottom-up process, simple building blocks self-assemble to form large and more complex supramolecular assemblies. In the molecular self-assembly process, molecules spontaneously interact with each other through noncovalent bonds, such as Van der Waals interactions, hydrogen bonds, aromatic interactions and electrostatic interactions, to form well-ordered ultrastructures. In recent years there is a great interest in the fabrication of new materials using natural building blocks and combining them in artificial systems, when proteins and peptides are of special interest due to their biological and chemical diversity as well as structural simplicity.

As has been previously shown by some of the inventors of the present invention, interactions between aromatic units play a central role in molecular self-assembly of amino acids [11-14]. These are attractive non-covalent interactions between planar aromatic rings which are referred as π-stacking. Aromatic interactions are made up of a combination of forces including electrostatic, hydrophobic and Van der Waals interactions. A major characteristic of aromatic interactions is their specific geometries, which endow them with specific properties, enabling recognition and selectivity. Reductionist approaches have shown that aromatic tetrapeptide fragments self-assemble to form amyloid-like structures [15]. In addition, the core recognition motif of the β-amyloid polypeptide, which plays a key role in Alzheimer disease, the diphenylalanine, has been shown to form ordered tubular and spherical structures [14]. Later studies reveled that other aromatic homodipeptides could form various structures at the nano-scale, including nanotubes, nanospheres, fibrillar assemblies, nano-plates and hydrogels.

U.S. Pat. No. 7,786,086 to some of the inventors of the present invention discloses a nanostructure composed of a plurality of peptides, each peptide containing at least one aromatic amino acid, whereby one or more of these peptides is end-capping modified and wherein the nanostructure can take a tubular, fibrillar, planar or spherical shape, and can encapsulate, entrap or be coated by other materials.

U.S. Patent Application No. 2009/0175785 to some of the inventors of the present invention is directed to novel peptide-based hydrogels, composed of short aromatic peptides (e.g., homodipeptides of aromatic amino acid residues).

EP Patent No. 1575867 to some of the inventors of the present invention discloses a tubular or spherical nanostructure composed of a plurality of peptides, wherein each of the plurality of peptides includes no more than 4 amino acids and whereas at least one of the 4 amino acids is an aromatic amino acid.

The major disadvantage of presently known bioadhesive materials, mimicking mussel adhesive properties, is the uncontrollable presentation of the functional adhesive sites relatively to the surface. In order to exploit the bioadhesive properties of nonoxidized DOPA-functionalized materials, it is important to have control over the presentation of the functional adhesive sites relative to the surface. Currently known DOPA-functional synthetic polymers expose the adhesive groups randomly, which reduces the adhesive properties of DOPA-functionalized materials.

The modification of polymers with catechol groups draws significant attention beyond adhesion due to the abilities of these groups to act as antioxidant agents, radical trappers, metal chelators, oxidizable reducing agents, etc. [25, 26]. Moreover, catechol redox chemistry was also utilized to form polymer-coated metal nanoparticles and mussel-inspired silver-releasing antibacterial hydrogels [27]. Although this approach has been successfully used for various important applications, one major challenge remains: the ability to present a high density of catechol functional groups in a defined ultrastructural organization and architecture at the nano-scale.

There exists, therefore, an unmet need for bioadhesive, anti-oxidant and/or antibacterial agents having a well-ordered structures with highly-oriented functional groups.

### SUMMARY OF THE INVENTION

The present invention provides self-assembled micro- and nano-structures, having an ordered structure with controllable orientation of sites that possess at least one of adhesive, anti-bacterial, anti-fouling and/or anti-oxidant properties, or any combination thereof. The micro- and nano-structures of the present invention provide superior adhesive, anti-bacterial anti-fouling and/or anti-oxidant properties as compared to currently known products, and they are biocompatible, thus finding utility in a variety of pharmaceutical, cosmetic and medical devices applications.

The present invention is based in part on the concept of mimicking adhesive, anti-oxidant anti-fouling and/or anti-bacterial biological systems by incorporating DOPA functional groups in self-assembling amino acids or peptides, with the aim of harnessing the molecular self-assembly process to form well-ordered structures endowed with functional properties due to a dense display of the catecholic moieties. The ability of the aromatic amino acids or short aromatic peptides to self-assemble into ordered nanostructures, and the organization of end-capping groups such as 9-fluorenylmethoxycarbonyl (Fmoc)-modified amino acids/peptides into hydrogels of nano-scale order allowed for the design of short self-assembling building blocks containing DOPA and DOPA derivatives.

Thus, the current invention employs molecular self-assembly for generating micro-structures and nano-structures. More specifically, the present invention is based on the unexpected discovery that use of amino acids comprising catecholic moieties, or incorporation of such amino acids into self-assembled peptides provides adhesive, anti-microbial, anti-fouling and/or anti-oxidant function to the resulting, self-assembled micro- or nano-structure, and allows for the generation of a highly structured product with superior properties. The well-ordered micro- or nano-structure of said amino acids or peptides allows controlled orientation of active moieties relative to the target surface, enhancing the adhesive, anti-fouling, anti-microbial and/or anti-oxidant properties of the product. Spatial orientation of the catecholic moieties of the well-ordered self-assembled fibrillar micro- and nano-structures of the present invention is schematically depicted in FIG. 1. The depicted micro- and nano-structures provide a surface comprised of the controllably exposed catecholic groups.

One possible route of incorporating amino acids comprising catecholic moieties is generating peptides comprising such amino acids along with self-assembling protein motifs, such as, but not limited to, the aromatic core recognition motif of the β-amyloid polypeptide—the di-phenylalanine dipeptide. The diphenylalanine module efficiently self-assembles into discrete well-ordered nanotubes [14]. These aromatic dipeptide nanotubes (ADNT) are formed under mild conditions and possess high mechanical stability and strength. In addition, ADNT can be aligned in a controlled fashion both vertically and horizontally [15-17]. As contemplated herein, by conjugating an amino acid comprising a catecholic moiety (e.g., DOPA or a DOPA derivative) to the di-phenylalanine motif and its derivatives, or by substituting the phenylalanine moieties with one or more DOPA or DOPA derivative moieties, well-ordered nanotubes may be created, wherein the DOPA motif is displayed on the external wall of the tube. These nanotubes may further be aligned to provide larger ordered functional surface area. As demonstrated herein, the inventors have found that addition of at least one amino acid comprising a catecholic moiety to the diphenylalanine module, or substitution of the diphenylalanine module with at least one amino acid comprising a catecholic moiety (e.g., DOPA or a DOPA derivative), yielded self-assembled micro- and nano-structures having at least one of adhesive, anti-bacterial, anti-fouling and/or anti-oxidant properties.

One currently preferred embodiment of the present invention comprises substitution of one or more phenylalanine moieties with amino acids comprising catecholic moieties (e.g., DOPA or a DOPA derivative) in known peptide recognition motifs. As further demonstrated herein, it has been shown that substituting aromatic units in known peptide recognition motifs with amino acids comprising catecholic moieties, yields self-assembled micro- and nano-structures having at least one of adhesive, anti-bacterial, anti-fouling and/or anti-oxidant properties.

Alternatively, it was unexpectedly discovered that self-assembled bioadhesive micro-structure or nano-structure can be formed from single amino acids comprising a catecholic moiety (e.g., DOPA).

Thus, according to a first aspect, the present invention provides a self-assembled micro- or nano-structure comprising (i) a plurality of aromatic amino acids selected from 3,4-dihydroxyphenyl-L-alanine (DOPA) and a DOPA-derivative; or (ii) a plurality of peptides, each peptide comprising between 2 and 9 amino acids, at least one of which is an aromatic amino acid selected from 3,4-dihydroxyphenyl-L-alanine (DOPA) and a DOPA-derivative; or (iii) a combination of said amino acids and peptides; wherein said micro- or nano-structure has at least one property selected from bioadhesive, anti-oxidant, anti-fouling, anti-bacterial and any combination thereof. Each possibility represents a separate embodiment of the present invention.

According to some embodiments, the micro- or nano-structure is selected from the group consisting of a fibrillar microstructure/nanostructure, a tubular microstructure/nanostructure, a spherical microstructure/nanostructure and a ribbon-like microstructure/nanostructure. In one embodiment, the micro- or nano-structure does not exceed about 500 nm in diameter. In another embodiment, the micro- or nano-structure is at least about 1 nm in diameter. Each possibility represents a separate embodiment of the present invention.

According to some embodiments, each peptide in the plurality of peptides comprises between 2 and 7 amino acids. Currently preferred peptides comprise two amino acids (dipeptides), three amino acids (tripeptides) or five amino acids (pentapeptides). Each possibility represents a separate embodiment of the present invention.

According to some embodiments, each peptide in the plurality of peptides comprises a plurality of aromatic amino acids selected from 3,4-dihydroxyphenyl-L-alanine (DOPA), a DOPA-derivative and a combination thereof.

According to further embodiments, at least one peptide in the plurality of peptides is a 3,4-dihydroxyphenyl-L-alanine-(3,4-dihydroxyphenyl-L-alanine) (DOPA-DOPA) homodipeptide. According to further embodiments, the DOPA-DOPA homodipeptide can be a dipeptide per se, or it can be incorporated into the backbone of a longer peptide. Thus, according to some embodiments, at least one peptide in the plurality of peptides incorporates at least one 3,4-dihydroxyphenyl-L-alanine-(3,4-dihydroxyphenyl-L-alanine) (DOPA-DOPA) homodipeptide in the peptide backbone. Optionally, the DOPA-DOPA homopeptide further comprises at least one end-capping modified moiety at the C- or N-terminus, as defined herein, for example an Fmoc moiety.

In other embodiments, single amino acids (i.e., DOPA or Fmoc-DOPA) can also self-assemble into micro- or nano-structures. Accordingly, another embodiment of the present invention is directed to a self-assembled bioadhesive micro- or nano-structure comprising a plurality of aromatic amino acids selected from 3,4-dihydroxyphenyl-L-alanine (DOPA) and a DOPA-derivative.

In some embodiments, the present invention is directed to a self-assembled micro- or nano-structure comprising a combination of a plurality of single amino acids and a plurality of peptides, as described herein.

In some embodiments, at least one amino acid or peptide in the plurality of amino acids or peptides, preferably each amino acid or peptide in the plurality of amino acids or peptides, further comprises at least one amino acid capable of enhancing cohesion, enhancing adhesion of said peptide to a surface, or a combination thereof, thus rendering a bioadhesive micro- or nano-structure. Preferably, the amino acid is charged at neutral pH. In some embodiments, the amino acid comprises a positively charged side chain capable of ionically interacting with negatively charged surface, or a negatively charged side chain capable of ionically interacting with positively charged surface. In currently preferred embodiments, the amino acid is selected from the group consisting of lysine, lysine analogs (e.g., ornithine), arginine, aspartic acid, glutamic acid, and histidine.

A currently preferred amino acid for incorporation into the plurality of DOPA containing peptides is lysine. As demonstrated herein, it was unexpectedly found that incorporation of a lysine residue into the DOPA-containing peptide, or conjugating lysine to DOPA assemblies provides self-assembled structures with bioadhesive properties. Without wishing to be bound by any particular theory or mechanism of action, it is hypothesized that the incorporation of a lysine residue into the DOPA-containing amino acid/peptide assemblies contributes to cohesion and thus indirectly improve adhesion. Moreover, lysine residues may also contribute to adhesion via ionic bonding to negatively charged surfaces. Indeed, recent evidence suggested that oxidation of DOPA residues to DOPA-quinone or DOPA-semiquinone can lead to intermolecular cross-linking of the MAPs, either with other DOPA residues by a radical mechanism or with ε-amino group of lysine (Lys) residues [18]. Moreover, lysine is a common residue in MAPs and the DOPA-lysine motif was previously fused for the design of adhesive polymers [24].

According to some embodiments, the micro- or nano-structure of the present invention further comprises at least one additional amino acid, selected from the group consisting of naturally occurring amino acids, synthetic amino acids and combinations thereof. Each possibility represents a separate embodiment of the present invention.

According to some embodiments, at least one amino acid or peptide in the plurality of amino acids or peptides comprises at least one end-capping modified moiety at the C- or N-terminus, or a combination thereof. According to further embodiments, the end capping moiety is selected from the group consisting of an aromatic end capping moiety and a non-aromatic end-capping moiety. According to still further embodiments, the end-capping moiety comprises a labeling moiety.

According to some embodiments, aromatic end capping moiety is selected from the group consisting of 9-fluorenylmethyloxycarbonyl (Fmoc) and benzyloxycarbonyl (Cbz). According to additional embodiments, the non-aromatic end capping moiety is selected from the group consisting of acetyl and tert-butoxycarbonyl (Boc). Each possibility represents a separate embodiment of the present invention. Additional examples of end-capping amino acids include, but are not limited to, naphthalene (Nap) derivatives, phenothiazine (PTZ)], azobenzene (Azo), pyrene (Pyr), or cinnamoyl.

According to a certain embodiment, at least one of the plurality of amino acids or peptides is selected from the group consisting of Fmoc-DOPA, DOPA-DOPA, DOPA-Phe-Phe, Fmoc-DOPA-DOPA, Fmoc-DOPA-DOPA-Lys, Fmoc-Phe-Phe-DOPA-DOPA-Lys, Lys-Leu-Val-DOPA-DOPA-Ala-Glu, and Asp-DOPA-Asn-Lys-DOPA, as well as and derivatives of any of the foregoing comprising an end capping moiety, preferably an Fmoc moiety. Each possibility represents a separate embodiment of the present invention.

According to some embodiments, the micro- or nano-structure of the present invention is provided in the form of a hydrogel. According to further embodiments, the hydrogel is characterized by a storage modulus G1 ranging from ˜20 Pa to ˜5 kPa according to the final concentration of the peptide, at 1 Hz frequency, 0.7% strain.

According to additional embodiments, there is provided a method of generating the self-assembled micro- or nano-structure described herein, the method comprising the step of incubating a plurality of amino acids or peptides under conditions which favor formation of the micro- or nano-structure.

According to some embodiments, the micro- or nano-structure of the present invention is capable of reducing a metal ion to neutral metal atom, wherein the metal may be selected from the group consisting of silver, gold, copper, platinum, nickel and palladium. Each possibility represents a separate embodiment of the present invention.

According to some embodiments, the micro- or nano-structure of the present invention may be used in preparation of a pharmaceutical composition, a cosmetic composition, or a medical device (e.g., a medical sealant or adhesive such as an adhesive patch or band-aid). In other embodiments, the micro- and nano-structures of the present invention are applied as a coating (e.g., an adhesive coating) to an existing medical device. Other utilities include, but are not limited to a drug delivery vehicle, a 3D scaffold for cell growth, tissue adhesive for regenerative medicine, biological glue that is resilient to the shear forces of blood flow, anti-bacterial and anti-oxidant uses, or any combination of the foregoing.

According to a certain embodiment, the micro- or nano-structure of the present invention may be used in the preparation of biological glue.

According to other embodiments, the micro- or nano-structure of the present invention may be used in the preparation of a composition for combating bacteria or treating bacterial infections.

According to other embodiments, the micro- or nano-structure of the present invention may be used as an anti-oxidant, a radical trapper, a metal chelator, or an oxidizable reducing agent.

In some embodiments, the micro- or nano-structures of the present invention can be co-assembled with other self-assembled peptides that are known in the art, such as Fmoc-Phe-Phe Boc-Phe-Phe and Phe-Phe, or co-assembled with polypeptides, polysaccharides, polymers, or a combination thereof.

In some embodiments, the micro- or nano-structures of the present invention can be co-assembled with polysaccharides that are known in the art to form hydrogels. Non-limiting examples of such peptides are hyaluronic acid.

According to alternative embodiments, there is provided a pharmaceutical composition, a cosmetic composition, a medical device or a medical device coating, comprising the self-assembled bioadhesive micro- or nano-structure of the present invention. The pharmaceutical or cosmetic composition, or the device, may further comprise a pharmaceutically or cosmetically acceptable carrier and one or more additional excipients, which may vary depending on the nature of the composition or device.

According to additional embodiments, there is provided a kit for forming the self-assembled bioadhesive micro- or nano-structure of the present invention, the kit comprising (i) a plurality of aromatic amino acids selected from 3,4-dihydroxyphenyl-L-alanine (DOPA) and a DOPA-derivative; or a plurality of peptides, each peptide comprising between 2 and 9 amino acids, at least one of which is an aromatic amino acid selected from 3,4-dihydroxyphenyl-L-alanine (DOPA) and a DOPA-derivative; or a combination of said amino acids and peptides; and (ii) an aqueous solution, each being individually packaged within the kit, wherein the plurality of amino acids or peptides and the solution are selected such that upon contacting said plurality of peptides and said solution, said micro- or nano-structure is formed.

In other embodiments, the present invention relates to the use of micro- or nano-structure described herein, for the encapsulation of an agent selected from the group consisting of a therapeutically active agent, a diagnostic agent, a biological substance and a labeling moiety.

In other embodiments, the present invention relates to a composition comprising the micro- or nano-structure as described herein, and an agent selected from the group consisting of a therapeutically active agent, a diagnostic agent, a biological substance and a labeling moiety. The micro- or nano-structure may in some embodiments encapsulate the agent, or in other embodiments may be attached to said agent by any covalent or non-covalent interactions. The agent may be selected from the group consisting of therapeutically active agents, diagnostic agents, biological substances and labeling moieties, such as, but not limited to drugs, cells, proteins, enzymes, hormones, growth factors, nucleic acids, organisms such as bacteria, fluorescence compounds or moieties, phosphorescence compounds or moieties, and radioactive compounds or moieties.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1: Schematic representation of the self-assembled bioadhesive nanostructures, comprising catecholic moieties.

FIG. 2A-2B: DOPA containing self-assembling peptides form ordered ultrastructures. (FIG. 2A) Chemical structure of the DOPA-containing designed peptides. (FIGS. 2B-2C) TEM micrographs of DOPA-DOPA dipeptide assemblies; (FIGS. 2D-2F) TEM micrographs of the hydrogel-forming Fmoc-DOPA-DOPA assemblies; (FIG. 2G) E-SEM micrographs of the Fmoc-DOPA-DOPA hydrogel after gradual dehydration.

FIGS. 3A-3F: Morphology characterization of Fmoc-DOPA-DOPA. FIGS. 3A-3B TEM and FIGS. 3C-3D: HR-SEM images of Fmoc-DOPA-DOPA, taken 24 hours after assembly, exhibiting tangled fibrous structures. FIGS. 3E-3F: HR-SEM images taken 2 minutes after the assembly, exhibiting large aggregates. Scale bar for the images is: 3A, 3C: 1 μm; 3B: 200 nm; 3D: 100 nm; 3E, 3F: 10 μm.

FIGS. 4A-4F: Rheological and structural properties of the Fmoc-DOPA-DOPA hydrogelator. Strain sweep (FIG. 4A) and frequency sweep (FIG. 4B) characterization of 5 mgmL⁻¹ in situ-formed hydrogel at 25° C.; (FIG. 4C) Gelation kinetics of Fmoc-DOPA-DOPA at different concentrations at 25° C.; (FIG. 4D) Gelation kinetics of 5 mgmL⁻¹ Fmoc-DOPA-DOPA at different temperatures; (FIG. 4E) Kinetics of absorbance at 405 nm at two concentrations and macroscopic visualization of the preparation; (FIG. 4F) HR-SEM micrographs of the turbid peptide solution immediately after inducing the assembly process (left and center panels) and of the semi-transparent gel after 2 h of incubation (right panel).

FIGS. 5A-5D: AFM measurements of Fmoc-DOPA-DOPA. Measurements were conducted on 2 mg/ml Fmoc-DOPA-DOPA (FIG. 5A) or 5 mg/ml Fmoc-DOPA-DOPA (FIG. 5B). Samples were imaged using tapping mode (40×40) and force/distance curves were determined at several points (three repeats at each point). The measured adhesive forces between the tip and the sample, represented by the minimum value of the curves, are summarized in the tables (FIGS. 5C-5D) for each of the samples.

FIGS. 6A-6B: Adhesion Force Map on Glass Slide (FIG. 6A). Adhesion Histogram from Force Map (FIG. 6B), the mean force is 7±1.2 nN.

FIGS. 7A-7C: AFM characterization of 5 mg/ml Fmoc-DOPA-DOPA prepared in 5% ethanol solution: (FIG. 7A) AFM image (tapping mode, scan size of 3×3 μm2) of the dried hydrogel reveal the presence of bulk fibrous structures. AFM image was obtained using ultra sharp MicroMasch AFM probe. (FIG. 7B) Adhesion force map and the corresponding histogram (FIG. 7C) of the sample, reveal mean force of 36±11 nN. Force data was obtained using SiO2 colloidal probe. Tip velocity 1000 nm/s. Compressive force 20 nN.

FIGS. 8A-8C: Silver reduction by pre-prepared Fmoc-DOPA-DOPA hydrogel. (FIG. 8A) Macroscopic visualization and UV-vis spectra of assemblies at 5 mgmL-1 taken after five days of incubation; (FIG. 8B) TEM micrographs of the formation of silver particles after one day of incubation of assemblies at 2.5 mgmL-1 (bottom panels) and a control gel with no addition of silver nitrate (upper panel); (FIG. 8C) TEM micrographs of the assemblies at 5 mgmL-1 after two days of incubation. The arrows indicate non-coated peptide assemblies. In all micrographs, negative staining was not applied.

FIGS. 9A-9C: DOPA-DOPA reaction with silver nitrate. HR-SEM images of 5 mg/ml DOPA-DOPA in the absence (FIG. 9A) or presence (FIGS. 9B-9C) of silver nitrate. Silver deposition was observed in the presence of silver nitrate. Scale bar for all images is 1 μm.

FIGS. 10A-10B: DOPA-DOPA reaction with silver nitrate, UV-visible extinction curves. Extinction values of 5 mg/ml DOPA-DOPA at different pH in the presence or absence of silver nitrate were measured between 250 to 800 nm, several hours after silver nitrate was added to the solution (FIG. 10A). In the presence of silver nitrate, a yellow color change resulting from a peak centered at ˜410 nm occurred due to silver formation (FIG. 10B—zoom-in).

FIGS. 11A-11D: AFM measurements of Fmoc-DOPA (5 mg/ml) at pH 5.5 Images of 5 mg/ml Fmoc-DOPA at pH 5.5 reveal sparse spheres at varied sizes as shown by AFM (FIGS. 11A-11B) and light microscopy (FIG. 11C). Force/distance curves were determined at several points (three repeats at each point). The measured adhesive forces between tip and sample, represented by the minimum value of the curves, are summarized in a table (FIG. 11D).

FIGS. 12A-12G: AFM measurements of 5 mg/ml Fmoc-DOPA. The experiment was conducted in water that were pre-adjusted to pH 8.7 by adding diluted NaOH solution. Images of Fmoc-DOPA (5 mg/ml) reveal a dense layer of spheres at varied sizes. Small spheres are observed at 3 μm×3 μm images (FIGS. 12A-12B), whereas larger spheres are observed at the 40 μm×40 μm images (FIGS. 12C-12D). Light microscopy image of the sample reveals a dense layer of spheres (FIG. 12E). Force/distance curves were determined at several points (three repeats at each point) for Fmoc-DOPA (5 mg/ml) in the absence (FIG. 12F) or in the presence (FIG. 12G) of Fe⁺³. The measured adhesive forces between tip and sample, represented by the minimum value of the curves, are summarized in a table for each of the samples.

FIGS. 13A-13D: AFM measurements of Fmoc-DOPA (2 mg/ml). The experiment was conducted in water, and the pH was pre-adjusted to pH 8.7 by adding diluted NaOH solution. Images of Fmoc-DOPA (2 mg/ml) reveal sparse spheres at varied sizes, as shown by AFM (FIGS. 13A-13B) and light microscopy (FIG. 13C). The 3D image of the sample indicates that the large spheres observed are truncated. Force/distance curves were determined at several points (three repeats at each point). The measured adhesive forces between tip and sample, represented by the minimum value of the curves, are summarized in a table (FIG. 13D).

FIGS. 14A-14C: AFM characterization of 1 mg/ml Fmoc-DOPA prepared in 1% ethanol solution, after one day of assembly: (FIG. 14A) AFM image (tapping mode, scan size of 10 μm×10 μm) reveal the presence of long fibers. AFM image was obtained using ultra sharp MicroMasch AFM probe. (FIG. 14B) Adhesion force map and the corresponding histogram (FIG. 14C) of the sample, reveal mean force of 31±10.6 nN. Tip velocity 1000 nm/s. Compressive force 20 nN.

FIGS. 15A-15E: Characterization of Fmoc-DOPA-DOPA-Lys assemblies. (FIG. 15A) Chemical structure and TEM analysis of 1.25 wt % Fmoc-DOPA-DOPA-Lys assemblies prepared in either 12.5% ethanol or 12.5% DMSO; (FIG. 15B) Adhesion force map and corresponding histogram of 1.25% wt Fmoc-DOPA-DOPA-Lys prepared in ethanol and water; (FIG. 15C) Adhesion force map and corresponding histogram of 1.25% wt Fmoc-DOPA-DOPA-Lys prepared in DMSO and water; (FIG. 15D) AFM images of the exposed area of the bottom (left and center panels) and top (right panel) glass surfaces after peeling two glass slides that were adhered overnight by an aliquot of 1.25% wt Fmoc-DOPA-DOPA-Lys in 12.5% ethanol; (FIG. 15E) AFM images of the exposed area of the bottom (left and center panels) and top (right panel) glass surfaces after peeling two glass slides that were adhered overnight by a preparation of 1.25% wt Fmoc-DOPA-DOPA-Lys in 12.5% DMSO.

FIGS. 16A-16F: Morphology characterization of DOPA-Phe-Phe at low HFIP concentrations. TEM images of DOPA-Phe-Phe (2 mg/ml) (FIGS. 16A-16C) or DOPA-Phe-Phe (5 mg/ml) (FIGS. 16D-16F) display sphere-like structures. Scale bars for the images are: 16a: 100 nm; 16b: 200 nm; 16c: 1 μm; 16d: 100 nm; 16e: 100 nm; 16f: 200 nm.

FIGS. 17A-17G: Morphology characterization of horizontally aligned DOPA-Phe-Phe. SEM (FIGS. 17A-17B) and HR-SEM (FIGS. 17C-17G) images of the horizontally aligned peptide ribbon-like structures. DOPA-Phe-Phe 50 mg/ml (FIGS. 17A-17B) or 100 mg/ml (FIGS. 17A-17G), were dissolved in 100% HFIP and deposited on a glass slide allowing rapid evaporation of the solvent, leading to structure alignment. Scale bar for the images is: 17A: 20 μm; 17B: 5 μm; 17C: 1 μm; 17D: 1 μm; 17E: 1 μm; 17F: 100 nm; 17G: 100 nm.

FIGS. 18A-18D: AFM measurements of 100 mg/ml DOPA-Phe-Phe. Images of 100 mg/ml DOPA-Phe-Phe dissolved in 100% HFIP reveal crowded arrangements of vertically aligned structures due to vapor deposition, as shown by AFM (FIGS. 18A-18B) and light microscopy (FIG. 18C). Force/distance curves were determined at several points (three repeats at each point). The measured adhesive forces between tip and sample, represented by the minimum value of the curves, are summarized in a table (FIG. 18D).

FIGS. 19A-19F: AFM measurements of DOPA-Phe-Phe (50 mg/ml). Images of 50 mg/ml DOPA-Phe-Phe dissolved in 100% HFIP reveal disperse arrangements of structures, as shown by AFM (FIG. 19A) and light microscopy (FIG. 19C). The 3D projection of the sample (FIG. 19B) reveals a wall-like structure, with cratered terrain (FIGS. 19D-19E). Force/distance curves were determined at several points (three repeats at each point). The measured adhesive forces between tip and sample, represented by the minimum value of the curves, are summarized in a table (FIG. 19F).

FIGS. 20A-20B: Amino acid sequence of human calcitonin (hCT) (SEQ ID. No. 1). Underlined are residues 15-19, which form the minimal amyloidogenic recognition module of hCT; the chemical structure of the module appears below (FIG. 20B). The chemical structure of the hCT-inspired DOPA-containing pentapeptide, Asp-DOPA-Asn-Lys-DOPA. The catechol hydroxyl substituents appear in red (FIG. 20B).

FIGS. 21A-21C: High-resolution microscopy of fibrillar assemblies formed by 6 mM Asp-DOPA-Asn-Lys-DOPA in water. (FIGS. 21A-21B) TEM micrographs, negative staining was applied. Scale bars represent 2 μm and 100 nm (FIG. 21C) E-SEM micrograph, scale bar represents 1 μm.

FIGS. 22A-22C: Congo Red (CR) staining of Asp-DOPA-Asn-Lys-DOPA. 10 sample of 6 mM solution was stained with CR and examined by (FIG. 22A) polarized optical microscopy and by (FIG. 22B) fluorescence microscopy. Brightfield image corresponding to the fluorescence microscopy micrograph. Scale bars represents 100 μm (FIG. 22C).

FIGS. 23A-23B: Secondary structure analysis of Asp-DOPA-Asn-Lys-DOPA. (FIG. 23A) FTIR spectrum of dried 6 mM solution sample. The spectrum was analyzed by curve-fitting the second derivative of the amide I′ region. CD spectrum of 0.15 mM in water at 25° C. (FIG. 23B).

FIGS. 24A-24B: Temperature-dependent CD spectra of 0.15 mM Asp-DOPA-Asn-Lys-DOPA in water (FIG. 24A). The temperature was increased in a stepwise fashion from 18° C. to 90° C. then similarly decreased to 18° C. (FIG. 24B) Transmission FTIR spectra of a dried sample that was taken from the CD cuvette at the end of the experiment, a dried sample of the same solution kept at room temperature and a baseline of water traces only. Insets are respective TEM micrographs of the CD cuvette content at the end of the experiment (FIG. 24B1 insert) and the solution kept at room temperature (FIG. 24B2 insert). Negative staining was not applied. Scale bars of the insets represent 200 nm.

FIGS. 25A-25C: TEM micrographs of 6 mM Asp-DOPA-Asn-Lys-DOPA in water assembled at room temperature for four days. Solution aliquot sampled after additional overnight incubation at room temperature as control (FIG. 25A). Solution aliquot sampled after additional overnight incubation at 37° C. (FIG. 25B). The same solution aliquot presented in the previous panel, sampled after 8 h recovery at room temperature (FIG. 25C). For all samples, negative staining was not applied. Scale bars represent 2 μm.

FIGS. 26A-26B: Silver deposition on Asp-DOPA-Asn-Lys-DOPA fibrillar assemblies following centrifugation, resuspension with 2.17 mM AgNO₃ for 15 min and subsequent washing. TEM image (FIG. 26A) of 15 mM peptide, negative staining was not applied. Scale bar represents 200 nm (FIG. 26B) E-SEM image of 6 mM peptide. Scale bar represents 5 μm.

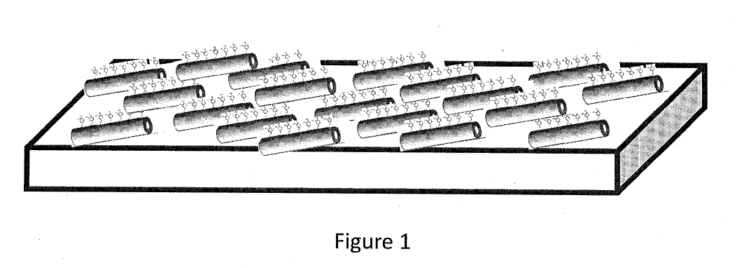

### DETAILED DESCRIPTION OF THE PRESENT INVENTION

The present invention provides self-assembled micro- and nano-structures, having an ordered structure with controllable orientation of adhesive, anti-microbial and/or anti-oxidant sites. The micro- and nano-structures of the present invention provide superior properties, e.g., at least one of adhesive and/or anti-microbial and/or anti-oxidant and/or antifouling properties as compared to currently known polymers, and they are biocompatible, thus finding utility in a variety of pharmaceutical, cosmetic and medical device applications.

Thus, according to one aspect of the present invention, the self-assembled bioadhesive micro- and nano-structures is provided, comprising a plurality of amino acids or peptides or a combination thereof, wherein each amino acid is an aromatic amino acid comprising a catecholic moiety, and/or wherein each peptide comprises at least one aromatic amino acid comprising a catecholic moiety. According to some embodiments, the at least one aromatic amino acid is selected from the group consisting of: 3,4-dihydroxyphenyl-L-alanine (DOPA), a DOPA-derivative and a combination thereof.

According to some aspects of the present invention, self-assembled micro- and nano-structures is provided, comprising (i) a plurality of aromatic amino acids selected from 3,4-dihydroxyphenyl-L-alanine (DOPA) and a DOPA-derivative; or (ii) a plurality of peptides, each peptide comprising between 2 and 9 amino acids, at least one of which is an aromatic amino acid selected from 3,4-dihydroxyphenyl-L-alanine (DOPA) and a DOPA-derivative; or (iii) a combination of said amino acids and peptides; wherein said micro- or nano-structure has at least one property selected from bioadhesive, anti-oxidant, anti-fouling, anti-bacterial and any combination thereof.

The term “DOPA derivative” as used herein refers to a chemical derivative of DOPA including, but not limited to, derivatization of any of the free functional groups of DOPA (i.e., carboxylic acid, amine or hydroxyl moieties). Examples of carboxylic acid derivatives include amides (—CONH₂) or esters (—COOR), wherein R is alkyl, trihaloalkyl, alkenyl, alkynyl, cycloalkyl, aryl or heteroaryl. Examples of amine functional groups include, e.g., N-acylated derivatives (NH—COR), wherein R is alkyl, trihaloalkyl, alkenyl, alkynyl, cycloalkyl, aryl or heteroaryl. Examples of hydroxyl function group include O-acylated derivatives (O—COR) or ether derivatives (OR) wherein R is alkyl, trihaloalkyl, alkenyl, alkynyl, cycloalkyl, aryl or heteroaryl. Each possibility represents a separate embodiment of the present invention. Derivatization of carboxylic acid moiety and/or amine moiety refers to the embodiments where DOPA is in the terminal position in the peptide (N-terminus or C-terminus).

According to some embodiments, at least one amino acid or peptide in the plurality of amino acids or peptides in the micro- or nano-structure of the present invention further comprises at least one additional amino acid capable of enhancing cohesion, enhancing adhesion of said peptide to a surface, or a combination thereof, rendering a bioadhesive micro- or nano-structure. Preferably, the amino acid is charged at neutral pH. In some embodiments, the amino acid comprises a positively charged side chain capable of ionically interacting with negatively charged surface, or a negatively charged side chain capable of ionically interacting with positively charged surface. In currently preferred embodiments, the amino acid is selected from the group consisting of lysine, lysine analogs (e.g., ornithine), arginine, aspartic acid, glutamic acid, and histidine. A currently preferred amino acid for incorporation into the plurality of DOPA-containing peptides is lysine.

According to additional embodiments, the micro- or nano-structure does not exceed about 50 μm in diameter, preferably does not exceed about 1 μm in diameter. According to further embodiments, the micro- or nano-structure does not exceed about 500 nm in diameter. According to still further embodiments, the micro- or nano-structure is at least 1 nm in diameter, e.g., about 1-50 nm, about 4-40 nm, about 10-30 nm, and the like. Each possibility represents a separate embodiment of the present invention.

In some embodiments, Fmoc-DOPA sphere diameter size range between about 20 nm to several microns (no more then 5), and the fiber width was less than about 20 nm. In other embodiments, the Fmoc-DOPA-DOPA fiber width was about 4-30 nm. In other embodiments, the DOPA-DOPA fiber width was about 20 to 50 nm Each possibility represents a separate embodiment of the present invention.

In some embodiments, the micro- or nano-structures of the present invention can be co-assembled with other self-assembled peptides that are known in the art. Examples of such self-assembled peptides are disclosed in U.S. Pat. No. 7,786,086, US 2009/0175785 and EP 1,575,867, the contents of each of which are incorporated by reference herein. Examples of known self-assembled peptides that can be combined or co-assembled with the DOPA-containing peptides of the present invention are peptide-based hydrogels, composed of short aromatic peptides (e.g., homodipeptides of aromatic amino acid residues). The aromatic amino acid residues comprise, for example, an aromatic moiety selected from the group consisting of substituted or unsubstituted naphthalenyl, substituted or unsubstituted phenanthrenyl, substituted or unsubstituted anthracenyl, substituted or unsubstituted [1,10]phenanthrolinyl, substituted or unsubstituted [2,2′]bipyridinyl, substituted or unsubstituted biphenyl and substituted or unsubstituted phenyl, including, but not limited to: polyphenylalanine peptides, polytriptophane peptides, and the like. Non-limiting examples include phenylalanine-phenylalanine dipeptide, naphthylalanine-naphthylalanine dipeptide, phenanthrenylalanine-phenanthrenylalanine dipeptide, anthracenylalanine-anthracenylalanine dipeptide, [1,10]phenanthrolinylalanine-[1,10]phenanthrolinylalanine dipeptide, [2,2′]bipyridinylalanine-[2,2′]bipyridinylalanine dipeptide, (pentahalo-phenylalanine)-(pentahalo-phenylalanine) dipeptide (including pentafluro phenylalanine, pentaiodo phenylalanine, pentabromo phenylalanine, and pentachloro phenylalanine dipeptides), phenylalanine-phenylalanine dipeptide, (amino-phenylalanine)-(amino-phenylalanine) dipeptide, (dialkylamino-phenylalanine)-(dialkylamino-phenylalanine) dipeptide, (halophenylalanine)-(halophenylalanine) dipeptide, (alkoxy-phenylalanine)-(alkoxy-phenylalanine) dipeptide, (trihalomethyl-phenylalanine)-(trihalomethyl-phenylalanine) dipeptide, (4-phenyl-phenylalanine)-(4-phenyl-phenylalanine) dipeptide and (nitro-phenylalanine)-(nitro-phenylalanine) dipeptide. Each of said peptides can further comprise an end-capping moiety as described herein. Specific examples of suitable peptides to be co-assembled with the peptides of the present invention include Fmoc-Phe-Phe, Phe-Phe (wherein Phe is phenylalanine), and halo-derivatives thereof, and the like.

According to some embodiments, the micro- or nano-structure of the present invention further comprises at least one additional amino acid, selected from the group consisting of naturally occurring amino acids, synthetic amino acids and combinations thereof. In other embodiments, all of the amino acids in the micro- or nano-structures of the present invention comprise catecholic moieties.

As used herein in the specification and in the claims section below the term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

According to some embodiments, the micro- or nano-structure of the present invention further comprises at least one naturally occuring amino acid, selected from the group consisting of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine and valine. Each possibility represents a separate embodiment of the invention.

According to some embodiments, the micro- or nano-structure of the present invention further comprises at least one non-conventional or modified amino acid, selected from the group consisting of halo-phenylalanine (including fluoro-phenylalanine, bromo-phenylalanine, iodo-phenylalanine, chloro-phenylalanine, pentafluro phenylalanine, pentaiodo phenylalanine, pentabromo phenylalanine, and pentachloro phenylalanine), phenylglycine, α-aminobutyric acid, α-amino-α-methylbutyrate, aminocyclopropane-carboxylate, aminoisobutyric acid, aminonorbornyl-carboxylate, cyclohexylalanine, cyclopentylalanine, D-alanine, D-arginine, D-aspartic acid, D-cysteine, D-glutamine, D-glutamic acid, D-histidine, D-isoleucine, D-leucine, D-lysine, D-methionine, D-ornithine, D-phenylalanine, D-proline, D-serine, D-threonine, D-tryptophan, D-tyrosine, D-valine, D-α-methylalanine, D-α-methylarginine, D-α-methylasparagine, D-α-methylaspartate, D-α-methylcysteine, D-α-methylglutamine, D-α-methylhistidine, D-α-methylisoleucine, D-α-methylleucine, D-α-methyllysine, D-α-methylmethionine, D-α-methylornithine, D-α-methylphenylalanine, D-α-methylproline, D-α-methylserine, D-α-methylthreonine, D-α-methyltryptophan, D-α-methyltyrosine, D-α-methylvaline, D-α-methylalnine, D-α-methylarginine, D-α-methylasparagine, D-α-methylasparatate, D-α-methylcysteine, D-N-methylleucine, D-N-methyllysine, N-methylcyclohexylalanine, D-N-methylornithine, N-methylglycine, N-methylaminoisobutyrate, N-(1-methylpropyl)glycine, N-(2-methylpropyl)glycine, N-(2-methylpropyl)glycine, D-N-methyltryptophan, D-N-methyltyrosine, D-N-methylvaline, γ-aminobutyric acid, L-t-butylglycine, L-ethylglycine, L-homophenylalanine, L-α-methylarginine, L-α-methylaspartate, L-α-methylcysteine, L-α-methylglutamine, L-α-methylhistidine, L-α-methylisoleucine, D-N-methylglutamine, D-N-methylglutamate, D-N-methylhistidine, D-N-methylisoleucine, D-N-methylleucine, D-N-methyllysine, N-methylcyclohexylalanine, D-N-methylornithine, N-methylglycine, N-methylaminoisobutyrate, N-(1-methylpropyl)glycine, N-(2-methylpropyl)glycine, D-N-methyltryptophan, D-N-methyltyrosine, D-N-methylvaline, γ-aminobutyric acid, L-t-butylglycine, L-ethylglycine, L-homophenylalanine, L-α-methylarginine, L-α-methylaspartate, L-α-methylcysteine, L-α-methylglutamine, L-α-methylhistidine, L-α-methylisoleucine, L-α-methylleucine, L-α-methylmethionine, L-α-methylnorvaline, L-α-methylphenylalanine, L-α-methylserine, L-α-methylvaline, L-α-methylleucine, N—(N-(2,2-diphenylethyl)carbamylmethyl-glycine, 1-carboxy-1-(2,2-diphenylethylamino)cyclopropane, L-N-methylalanine, L-N-methylarginine, L-N-methylasparagine, L-N-methylaspartic acid, L-N-methylcysteine, L-N-methylglutamine, L-N-methylglutamic acid, L-N-methylhistidine, L-N-methylisolleucine, L-N-methylleucine, L-N-methyllysine, L-N-methylmethionine, L-N-methylnorleucine, L-N-methylnorvaline, L-N-methylornithine, L-N-methylphenylalanine, L-N-methylproline, L-N-methylserine, L-N-methylthreonine, L-N-methyltryptophan, L-N-methyltyrosine, L-N-methylvaline, L-N-methylethylglycine, L-N-methyl-t-butylglycine, L-norleucine, L-norvaline, α-methyl-aminoisobutyrate, α-methyl-γ-aminobutyrate, α-methylcyclohexylalanine, α-methylcyclopentylalanine, α-methyl-α-napthylalanine, α-methylpenicillamine, N-(4-aminobutyl)glycine, N-(2-aminoethyl)glycine, N-(3-aminopropyl)glycine, N-amino-α-methylbutyrate, α-napthylalanine, N-benzylglycine, N-(2-carbamylethyl)glycine, N-(carbamylmethyl)glycine, N-(2-carboxyethyl)glycine, N-(carboxymethyl)glycine, N-cyclobutylglycine, N-cycloheptylglycine, N-cyclohexylglycine, N-cyclodecylglycine, N-cyclododeclglycine, N-cyclooctylglycine, N-cyclopropylglycine, N-cycloundecylglycine, N-(2,2-diphenylethyl)glycine, N-(3,3-diphenylpropyl)glycine, N-(3-indolylyethyl)glycine, N-methyl-γ-aminobutyrate, D-N-methylmethionine, N-methylcyclopentylalanine, D-N-methylphenylalanine, D-N-methylproline, D-N-methylserine, D-N-methylserine, D-N-methylthreonine, N-(1-methylethyl)glycine, N-methyla-napthylalanine, N-methylpenicillamine, N-(p-hydroxyphenyl)glycine, N-(thiomethyl)glycine, penicillamine, L-α-methylalanine, L-α-methylasparagine, L-α-methyl-t-butylglycine, L-methylethylglycine, L-α-methylglutamate, L-α-methylhomophenylalanine, N-(2-methylthioethyl)glycine, N-(3-guanidinopropyl)glycine, N-(1-hydroxyethyl)glycine, N-(hydroxyethyl)glycine, N-(imidazolylethyl)glycine, N-(3-indolylyethyl)glycine, N-methyl-γ-aminobutyrate, D-N-methylmethionine, N-methylcyclopentylalanine, D-N-methylphenylalanine, D-N-methylproline, D-N-methylserine, D-N-methylthreonine, N-(1-methylethyl)glycine, N-methyla-napthylalanine, N-methylpenicillamine, N-(p-hydroxyphenyl)glycine, N-(thiomethyl)glycine, penicillamine, L-α-methylalanine, L-α-methylasparagine, L-α-methyl-t-butylglycine, L-methylethylglycine, L-α-methylglutamate, L-α-methylhomophenylalanine, N-(2-methylthioethyl)glycine, L-α-methyllysine, L-α-methylnorleucine, L-α-methylornithine, L-α-methylproline, L-α-methylthreonine, L-α-methyltyrosine, L-N-methylhomophenylalanine, and N—(N-(3,3-diphenylpropyl)carbamylmethyl(1)glycine. Each possibility represents a separate embodiment of the invention.

The term “peptide” as used herein refers to a plurality of amino acids (at least two), and encompasses native peptides (including degradation products, synthetically synthesized peptides or recombinant peptides) and peptidomimetics (typically, synthetically synthesized peptides), as well as peptoids and semipeptoids which are peptide analogs.

The term “amino acid comprising a catecholic moiety” refers to, e.g., DOPA or a DOPA derivative as defined herein.

The micro- and nano-structures obtained by incorporating a catecholic moiety comprising amino acid into the well-known self-assembly peptide motifs were characterized and found to be in various structural forms, such as, but not limited to, fibrilar, tubular, spherical and ribbon-like structures, and any combination thereof.

As used herein, the term “nano-structure” refers to a physical structure, which in at least one dimension has a size ranging from about 1 nm to less than about 1,000 nm, for example about 10 nm or about 20 nm or about 50 nm to about 100 nm or about 200 nm or about 500 or less than about 1,000 nm.

As used herein, the term “micro-structure” refers to a physical structure, which in at least one dimension has a size ranging from about 1 μm to about 100 μm, for example about 10 μm or about 20 μm or about 50 μm to about 100 μm.

As used herein the phrase “tubular or spherical micro- or nano-structure” refers to a spherical or elongated tubular or conical structure having a diameter or a cross-section of less than about 50 μm (spherical structure) or less than about 500 nm (tubular structure). The length of the tubular micro- or nano-structure of the present invention is at least about 1 μm. It will be appreciated, though, that the tubular structure of the present invention can be of infinite length (i.e., macroscopic fibrous structures) and as such can be used in the fabrication of hyper-strong materials.

As used herein the phrase “fibrillar nano-structure” refers to a filament or fiber having a diameter or a cross-section of less than about 100 nm. The length of the fibrillar nanostructure of the present invention is preferably at least about 1 μm. It will be appreciated, though, that the fibrillar structure of the present invention can be of infinite length (i.e., macroscopic fibrous structures) and as such can be used in the fabrication of hyper-strong materials.

As used herein the phrase “ribbon-like nano-structure” refers to a filament or fiber, packed in a flat ribbon-like structure, having a diameter or a cross-section of less than about 500 nm. The length of the ribbon-like nano-structure of the present invention is preferably at least 1 about μm. It will be appreciated, though, that the ribbon-like structure of the present invention can be of infinite length (i.e., macroscopic fibrous structures) and as such can be used in the fabrication of hyper-strong materials. Preferably, the ribbon-like nano-structures described herein are characterized as non-hollowed or at least as having a very fine hollow.

In some embodiments, the micro- or nano-structure of the present invention is further characterized by adhesive properties. Adhesion of the micro- and nano-structures to glass was measured by atomic force microscopy (AFM), as described in the following Examples. According to some embodiments, the shear strength of the micro- and nano-structures on glass is from about 2 kPa to about 15 kPa.

In some embodiments of the present invention, the self-assembled micro- or nano-structure may comprise a plurality of amino acids comprising a catecholic moiety (e.g., DOPA or a DOPA derivative such as Fmoc-DOPA). In other embodiments, the self-assembled micro- or nano-structure may comprise a plurality of peptides, each comprising between 2 and 9 amino acids. According to a certain embodiment of this aspect of the present invention, each peptide in said plurality of peptides comprises between 2 and 8 amino acids. According to a certain embodiment of this aspect of the present invention, each peptide in said plurality of peptides comprises between 2 and 7 amino acids. According to a certain embodiment of this aspect of the present invention, each peptide in said plurality of peptides comprises between 2 and 6 amino acids. According to a certain embodiment of this aspect of the present invention, each peptide in said plurality of peptides comprises between 2 and 5 amino acids. According to a certain embodiment of this aspect of the present invention, each peptide in said plurality of peptides comprises between 2 and 4 amino acids. According to a certain embodiment of this aspect of the present invention, each peptide in said plurality of peptides comprises between 2 and 3 amino acids. In currently preferred embodiments, at least one peptide comprises two amino acids (dipeptide), three amino acids (tripeptides) or five amino acids (pentapeptide). In each of the aforementioned peptides, at least one amino acid comprising a catecholic moiety (e.g., DOPA or a DOPA derivative such as Fmoc-DOPA) is present.

In some embodiments, amino acid comprising a catecholic moiety is located within the peptide sequence, such as, but not limited to, Asp-DOPA-Asn-Lys-DOPA, Lys-Leu-Val-DOPA-DOPA-Ala-Glu, Fmoc-Phe-Phe-DOPA-DOPA-Lys, DOPA-DOPA-Lys, and derivatives thereof further comprising an end-capping moiety, for example Fmoc.

In other embodiments, the amino acids or peptides are incorporated into a hybrid structure comprising the amino acids or peptides comprising a catecholic moiety, in combination with other amino acids or peptides in varying molar ratios. Non-limiting examples of such hybrid structures include Fmoc-DOPA-DOPA+Fmoc-Lys; Fmoc-DOPA-DOPA+Fmoc-Phe-Phe; Fmoc-DOPA-DOPA+Lys; and Fmoc-DOPA-DOPA+DOPA. Each possibility represents a separate embodiment of the present invention.

According to some embodiments, at least one of the peptides in the plurality of peptides is a dipeptide. According to further embodiments, the dipeptide is a homodipeptide. According to a preferred embodiment, the homodipeptide is DOPA-DOPA dipeptide, which may be a homodipeptide per se, or may be incorporated into a longer peptide backbone. In accordance with this embodiment, a plurality of DOPA-DOPA homodipeptides surprisingly formed tangled fibril-like structure, in contrast to Phe-Phe homodipeptides, which typically self-assemble into tubular structures.

According to further embodiments, at least one of the peptides in said plurality of peptides is a tripeptide. According to further embodiments, the tripeptide incorporates a homodipeptide in the tripeptide backbone. According to some embodiments, the tripeptide incorporates a DOPA-DOPA homopeptide in the backbone thereof.

According to other embodiments, the peptides which form the micro- and nano-structures of the present invention further incorporate at least one aromatic amino acid comprising substituted or unsubstituted naphthalenyl and substituted or -, halo, nitro, azo, hydroxy, alkoxy, thiohydroxy, thioalkoxy, cyano, and amine.

In one preferred embodiment, the present invention includes the use of tripeptides including one aromatic amino acid comprising a catecholic moiety, and a homodipeptide comprising aromatic moieties.

Some non-limiting examples of homodipeptides, which can be incorporated in the peptide backbone together with an aromatic amino acid comprising a catechol moiety (e.g., DOPA), include phenylalanine-phenylalanine, (amino-phenylalanine)-(amino-phenylalanine), (dialkylamino-phenylalanine)-(dialkylamino-phenylalanine), halophenylalanine-halophenylalanine, (alkoxy-phenylalanine)-(alkoxy-phenylalanine), (trihalomethyl-phenylalanine)-(trihalomethyl-phenylalanine), (4-phenyl-phenylalanine)-(4-phenyl-phenylalanine), (nitro-phenylalanine)-(nitro-phenylalanine), naphthylalanine-naphthylalanine, anthracenylalanine-anthracenylalanine, [1,10]phenanthrolinylalanine,-[1,10]phenanthrolinylalanine, [2,2′]bipyridinylalanine-[2,2′]bipyridinylalanine, (4-phenyl phenylalanine)-(4-phenyl phenylalanine) and (p-nitro-phenylalanine)-(p-nitro-phenylalanine). According to the preferred embodiment, the tripeptide DOPA-Phe-Phe is used.

The micro- and nano-structures of the present invention, comprising an aromatic homodipeptide, such as Phe-Phe and an aromatic amino acid comprising a catecholic moiety, were surprisingly found to self-assemble into sphere particles or ribbon-like micro- and nano-structures, in contrast to Phe-Phe homodipeptides, which typically self-assemble into tubular structures.

The present invention also encompasses micro- and nano-structures comprising a plurality of longer peptides, wherein at least one of the plurality of peptides comprises 4-9 amino acids, preferably 4-7 amino acids. According to some embodiments, each of the plurality of peptides comprises 4-9 amino acids, preferably 4-7 amino acids. Said peptides may be based on fragments of amyloidogenic proteins that were shown to form typical amyloid-like structures. The adhesive, anti-microbial, anti-oxidant and/or anti-fouling properties of said micro- and nano-structures are provided by substituting phenylalanine present in said fragments by at least one amino acid comprising a catecholic moiety (e.g., DOPA). According to some embodiments, said fragments further comprise lysine residue, which is another main constituent of mussel adhesive proteins. Lysine residue may contribute to adhesion via ionic bonding to negatively charged surfaces, and intermolecular cross-linking with o-quinones [18]. Some non-limiting examples of such proteins are Lys-Leu-Val-DOPA-DOPA-Ala-Glu and Asp-DOPA-Asn-Lys-DOPA. Lys-Leu-Val-DOPA-DOPA-Ala-Glu is based on Lys-Leu-Val-Phe-Phe-Ala-Glu heptapeptide fragment of the β-amyloid peptide associated with Alzheimer's disease (Aβ16-22) that was shown to form highly ordered amyloid fibrils and tubular structures [19-20]. Asp-DOPA-Asn-Lys-DOPA is based on Asp-Phe-Asn-Lys-Phe (SEQ ID. No. 2), a pentapeptide fragment derived from the human calcitonin (hCT) polypeptide hormone that was shown to form amyloid-like fibrils [15].

The present invention also envisages self-assembled bioadhesive micro- and nano-structures which are composed of a plurality of peptides being longer than the above described (e.g., 10-150 amino acids), wherein each peptide comprises at least one aromatic amino acid comprising a catecholic moiety (e.g., DOPA).

The micro- and nano-structures of the present invention may further comprise end-capping modified amino acids or peptides. Thus, according to an embodiment of the present invention, at least one of the plurality of peptides of the self-assembled bioadhesive micro- or nano-structure is modified by one or more aromatic end capping moiety. According to another embodiment, each of the plurality of peptides of the self-assembled bioadhesive micro- or nano-structure is modified by one or more aromatic end capping moiety. The peptides may further be modified by one or more non-aromatic end capping moiety.

The phrase “end-capping modified moiety”, as used herein, refers to an amino acid or peptide which has been modified at the N-(amine) terminus and/or the C-(carboxyl) terminus thereof. The end-capping modification refers to the attachment of a chemical moiety to the terminus, so as to form a cap. Such a chemical moiety is referred to herein as an end capping moiety and is typically also referred to herein and in the art, interchangeably, as a peptide protecting moiety or group.

Representative examples of aromatic end capping moieties suitable for N-terminus modification include, without limitation, fluorenylmethyloxycarbonyl (Fmoc), naphthalene (Nap) derivatives, phenothiazine (PTZ)], azobenzene (Azo), pyrene (Pyr), and cinnamoyl.

Representative examples of non-aromatic end capping moieties suitable for N-terminus modification include, without limitation, formyl, acetyl trifluoroacetyl, tert-butoxycarbonyl (Boc), trimethylsilyl, and 2-trimethylsilyl-ethanesulfonyl.

Representative examples of non-aromatic end capping moieties suitable for C-terminus modification include, without limitation, amides, allyloxycarbonyl, trialkylsilyl ethers and allyl ethers. Representative examples of aromatic end capping moieties suitable for C-terminus modification include benzyl, benzyloxycarbonyl (Cbz), trityl and substituted trityl groups.

According to several embodiments, micro- and nano-structures comprising end-capping modified amino acids or peptides include Fmoc-DOPA, Fmoc-DOPA-DOPA and Fmoc-DOPA-DOPA-Lys. According to further embodiments, the micro- and nano-structures comprising end-capping modified amino acids or peptides include Fmoc-Phe-Phe-DOPA-DOPA-Lys.

The end-capping modification changes the structure of the end-capping of the peptide, changing its chemical and physical properties and therefore changing the chemical and physical properties of the peptide and the chemical and physical properties of the resulting nanostructure. Using such end-capping modification, micro- and nano-structures in which these properties are finely controlled can be formed and hence, controlled fabrication of e.g., films, monolayer, or other macroscopic structures with nano-scale order is allowed.

As demonstrated in the following Examples section, it was found that aromatic amino acids comprising a catecholic moiety, or peptides comprising such amino acids, which are modified with an aromatic end-capping moiety, self-assembles into sphere-like particles or into a fibrillar micro- or nano-structures, having adhesive properties. The formation of bioadhesive fibrillar micro- and nano-structures was particularly observed while utilizing DOPA-DOPA homodipeptides modified by an aromatic end-capping moiety, similarly to Phe-Phe end-capping modified homodipeptides, and the formation of bioadhesive spheres was observed while utilizing Fmoc-DOPA building blocks.

As is further demonstrated and discussed in the Examples section that follows, it was found that the fibrillar nano-structure in addition to the adhesive property was characterized by macroscopic properties of a hydrogel, with storage modulus (G′) ranging that could be modulated with high dynamic range from ˜20 Pa to 5 kPa.

As the micro- and nano-structures of the present invention comprise an aromatic amino acid having a catecholic moiety, wherein a catechol group has redox properties, said micro- and nano-structures are capable of reducing a metal ion to neutral metal atom. This trait was previously utilized to form metal core-polymer shell nanoparticles [21] and mussel-inspired silver-releasing antibacterial hydrogels [22]. As demonstrated herein, it was found that the hydrogels of the present invention were capable of reducing silver nitrate to spontaneously form silver particles, as described in following Examples. As the reduction of metal ions is performed by the catechloic groups of the plurality of peptides, which are controllably presented upon the micro- or nano-structure, the orientation of metal nanoparticles may be accordingly controlled. Thus, the self-assembled micro- and nano-structures may comprise a metal, wherein said metal is at least partially enclosed by a discrete fibrillar micro- or nano-structure. The self-assembled micro- and nano-structures may further comprise a metal controllably deposited on the outer shell of the fibrillar nano-structure. The metals, which can be deposited on the micro- and nano-structures include, but are not limited to, silver, gold, copper, platinum, nickel and palladium.

Based on the redox properties of the micro- or nano-structures of the present invention, they are useful as an ant-oxidant composition. Furthermore, the micro- or nano-structures of the present invention may be used as a radical trapper, a metal chelator, or an oxidizable reducing agent. Alternatively, the micro- or nano-structures of the present invention may be used for preparing compositions for combating bacteria or treating bacterial infections. Each possibility represents a separate embodiment of the present invention.

The term “anti-bacterial” may refer to one or more of the following effects: killing the bacteria (bacteriocide), causing halt of growth of bacteria (bacteriostatic), prevention of bacterial infection, prevention of bio-film formation and disintegration of a formed biofilm, and decrease in bacterial virulence.

Examples of bacterial strain that can be treated/disinfected by the composition of the invention (both as a disinfecting composition and as a pharmaceutical composition) are all gram negative and gram positive bacteria and in particular pathogenic gram negative and gram positive bacteria.

The term “combating bacteria” or “treating bacterial infection” may refer to one of the following: decrease in the number of bacteria, killing or eliminating the bacteria, inhibition of bacterial growth (stasis), inhibition of bacterial infestation, inhibition of biofilm formation, disintegration of existing biofilm, or decrease in bacterial virulence.

In other embodiments of the present invention, fibrous network of the micro- and nano-structures, which have a form of a hydrogel, may contain microscopic hollow cavities. This structural feature indicates that such hydrogel can be utilized as a matrix for encapsulating or attaching various agents thereto. In addition, these hollow cavities further enable to entrap therein biological substances such as cells (e.g., neural cells), allowing expansion and elongation of the cells within the hydrogel.

Agents that can be beneficially encapsulated in or attached to the hydrogel include, for example, therapeutically active agents, diagnostic agents, biological substances and labeling moieties. More particular examples include, but are not limited to, drugs, cells, proteins, enzymes, hormones, growth factors, nucleic acids, organisms such as bacteria, fluorescence compounds or moieties, phosphorescence compounds or moieties, and radioactive compounds or moieties.

As used herein, the phrase “therapeutically active agent” describes a chemical substance, which exhibits a therapeutic activity when administered to a subject. These include, as non-limiting examples, inhibitors, ligands (e.g., receptor agonists or antagonists), co-factors, anti-inflammatory drugs (steroidal and non-steroidal), antipsychotic agents, analgesics, anti-thrombogenic agents, anti-platelet agents, anticoagulants, anti-diabetics, statins, toxins, antimicrobial agents, anti-histamines, metabolites, anti-metabolic agents, vasoactive agents, vasodilator agents, cardiovascular agents, chemotherapeutic agents, antioxidants, phospholipids, anti-proliferative agents and heparins.

As used herein, the phrase “biological substance” refers to a substance that is present in or is derived from a living organism or cell tissue. This phrase also encompasses the organisms, cells and tissues. Representative examples therefore include, without limitation, cells, amino acids, peptides, proteins, oligonucleotides, nucleic acids, genes, hormones, growth factors, enzymes, co-factors, antisenses, antibodies, antigens, vitamins, immunoglobulins, cytokines, prostaglandins, vitamins, toxins and the like, as well as organisms such as bacteria, viruses, fungi and the like.

As used herein, the phrase “diagnostic agent” describes an agent that upon administration exhibits a measurable feature that corresponds to a certain medical condition. These include, for example, labeling compounds or moieties, as is detailed hereinunder. As used herein, the phrase “labeling compound or moiety” describes a detectable moiety or a probe which can be identified and traced by a detector using known techniques such as spectral measurements (e.g., fluorescence, phosphorescence), electron microscopy, X-ray diffraction and imaging, positron emission tomography (PET), single photon emission computed tomography (SPECT), magnetic resonance imaging (MRI), computed tomography (CT) and the like.

Representative examples of labeling compounds or moieties include, without limitation, chromophores, fluorescent compounds or moieties, phosphorescent compounds or moieties, contrast agents, radioactive agents, magnetic compounds or moieties (e.g., diamagnetic, paramagnetic and ferromagnetic materials), and heavy metal clusters, as is further detailed hereinbelow, as well as any other known detectable moieties.

As used herein, the term “chromophore” refers to a chemical moiety or compound that when attached to a substance renders the latter colored and thus visible when various spectrophotometric measurements are applied. A heavy metal cluster can be, for example, a cluster of gold atoms used, for example, for labeling in electron microscopy or X-ray imaging techniques.

As used herein, the phrase “fluorescent compound or moiety” refers to a compound or moiety that emits light at a specific wavelength during exposure to radiation from an external source. As used herein, the phrase “phosphorescent compound or moiety” refers to a compound or moiety that emits light without appreciable heat or external excitation, as occurs for example during the slow oxidation of phosphorous.

As used herein, the phrase “radioactive compound or moiety” encompasses any chemical compound or moiety that includes one or more radioactive isotopes. A radioactive isotope is an element which emits radiation. Examples include α-radiation emitters, β-radiation emitters or γ-radiation emitters.

While a labeling moiety can be attached to the hydrogel, in cases where the one or more of the peptides composing the hydrogel is an end-capping modified peptide, the end-capping moiety can serve as a labeling moiety per se.

Thus, for example, in cases where the Fmoc group described hereinabove is used as the end-capping moiety, the end-capping moiety itself is a fluorescent labeling moiety. In another example, wherein the Fmoc described hereinabove further includes a radioactive fluoro atom (e.g., ¹⁸F) is used as the end-capping moiety, the end-capping moiety itself is a radioactive labeling moiety.

Other materials which may be encapsulated by the hydrogel of the present invention include, without limitation, conducting materials, semiconducting materials, thermoelectric materials, magnetic materials, light-emitting materials, biominerals, polymers and organic materials.

Each of the agents described herein can be attached to or encapsulated in the hydrogel by means of chemical and/or physical interactions. Thus, for example, compounds or moieties can be attached to the external and/or internal surface of the hydrogel, by interacting with functional groups present within the hydrogel via, e.g., covalent bonds, electrostatic interactions, hydrogen bonding, van der Waals interactions, donor-acceptor interactions, aromatic (e.g., π-π interactions, cation-π interactions and metal-ligand interactions. These interactions lead to the chemical attachment of the material to the peptide fibrous network of the hydrogel. As an example, various agents can be attached to the hydrogel via chemical interactions with the side chains, N-terminus or C-terminus of the peptides composing the hydrogel and/or with the end-capping moieties, if present.

Alternatively, various agents can be attached to the hydrogel by physical interactions such as magnetic interactions, surface adsorption, encapsulation, entrapment, entanglement and the likes.

The micro- and nano-structures of the present invention are preferably generated by allowing a highly concentrated aqueous solution of the peptides of the present invention to self-assemble under mild conditions as detailed in Example 1 of the Examples section which follows.

Alternatively, the preparation of the hydrogel can also be performed upon its application, such that the plurality of peptides and the aqueous solution are each applied separately to the desired site and the hydrogel is formed upon contacting the peptides and the aqueous solution at the desired site of application. Thus, for example, contacting the peptides and the aqueous solution can be performed in vivo, such that the plurality of peptides and the aqueous solution are separately administered.

According to these embodiments, the administration is preferably effected locally, into a defined bodily cavity or organ, where the plurality of peptides and the aqueous solution become in contact while maintaining the desired ratio therebetween that would allow the formation of a self-assembled bioadhesive nanostructure within the organ or cavity.

Using such a route of preparing the hydrogel in vivo allows to beneficially utilize the formed micro- or nano-structure in applications such as, for example, dental procedures, as a dental glue, dental implant or filling material, cosmetic applications, tissue regeneration, implantation, and in would healing, as a wound dressing that is formed at a bleeding site, as is further detailed hereinbelow.

Thus, according to another aspect of the present invention there is provided a kit for forming the micro- and nano-structures described herein which comprise a plurality of amino acids or peptides, as described herein and an aqueous solution, as described herein, each being individually packaged within the kit, wherein the plurality of peptides and the solution are selected such that upon contacting the plurality of peptides and the solution, a self-assembled bioadhesive nanostructure, as described herein, is formed.

Such a kit can be utilized to prepare the micro- and nano-structures described herein at any of the desired site of actions (e.g., a bodily cavity or organ) described hereinabove. The kit can be designed such that the plurality of specific peptides and the aqueous solution would be in such a ratio that would allow the formation of the desired micro- and nano-structure at the desired site of application.

As used herein, the phrases “desired site of application” and “desired application site” describe a site in which application of the micro- and nano-structures of the present invention is beneficial, namely, in which the micro- and nano-structures can be beneficially utilized for therapeutic, diagnostic, cosmetic, and/or biomedical applications, as described in detail hereinbelow. Such a kit can further comprise an active agent, as is detailed hereinbelow.

The active agent can be individually packaged within the kit or can be packaged along with the plurality of peptides or along with the aqueous solution.

By being remarkably adhesive and having a well-ordered structure, and further by being stable, biocompatible and capable of encapsulating therein of various agents, the micro- and nano-structures described herein can be beneficially utilized in various applications, as is detailed hereinunder.

The bioadhesive, anti-microbial and/or anti-oxidant self-assembled micro- and nano-structures described herein can, for example, form a part of pharmaceutical or cosmetic compositions, either alone or in the presence of a pharmaceutically or cosmetically acceptable carrier. The micro- and nano-structures described herein may further be used in medical devices (e.g., a medical sealant or adhesive). In other embodiments, the micro- and nano-structures of the present invention are applied as a coating (e.g., an adhesive coating) to an existing medical device.

As used herein, a “pharmaceutical or cosmetic composition” refers to a preparation of the micro- and nano-structures described herein, with other chemical components such as acceptable and suitable carriers and excipients. The purpose of a pharmaceutical composition is to facilitate administration of a compound to an organism. The purpose of a cosmetic composition is typically to facilitate the topical application of a compound to an organism, while often further providing the preparation with aesthetical properties. Hereinafter, the term “pharmaceutically, or cosmetically acceptable carrier” refers to a carrier or a diluent that does not cause significant irritation to an organism and does not abrogate the biological activity and properties of the applied compound. Examples, without limitations, of carriers include propylene glycol, saline, emulsions and mixtures of organic solvents with water, as well as solid (e.g., powdered) and gaseous carriers.

The compositions described herein may be formulated in conventional manner using one or more acceptable carriers comprising excipients and auxiliaries, which facilitate processing of the nanoparticles into preparations. Proper formulation is dependent upon the route of administration chosen. Techniques for formulation and administration of drugs may be found in “Remington's Pharmaceutical Sciences,” Mack Publishing Co., Easton, Pa., latest edition.

The pharmaceutical compositions described herein can be formulated for various routes of administration. Suitable routes of administration may, for example, include oral, sublingual, inhalation, rectal, transmucosal, transdermal, intracavemosal, topical, intestinal or parenteral delivery, including intramuscular, subcutaneous and intramedullary injections as well as intrathecal, direct intraventricular, intravenous, intraperitoneal, intranasal, or intraocular injections. Bioadhesive self-assembled compositions of the present invention are particularly useful for transdermal drug delivery systems, as bioadhesives incorporated into pharmaceutical formulations allow enhancing the drug absorption by mucosal cells and provide timed-release of the drug to the target mucosal site [23].

Formulations for topical administration include but are not limited to lotions, ointments, gels, creams, suppositories, drops, liquids, sprays and powders. Conventional carriers, aqueous, powder or oily bases, thickeners and the like may be necessary or desirable.

Compositions for oral administration include powders or granules, suspensions or solutions in water or non-aqueous media, sachets, capsules or tablets. Thickeners, diluents, flavorings, dispersing aids, emulsifiers or binders may be desirable. Formulations for parenteral administration may include, but are not limited to, sterile solutions which may also contain buffers, diluents and other suitable additives. Timed oral compositions are envisaged for treatment.

The micro- and nano-structures may be applied as a coating to a solid oral dosage formulation, such as a tablet or gel-capsule or to a transmucosal drug delivery device. The bioadhesive micro- and nano-structure may further be present in the matrix of a tablet or transmucosal drug delivery device. The micro- and nano-structures of the present invention may further encapsulate the drug or to function as a shell in the core-shell tablet or device, wherein the core comprises a drug to be delivered.

The medical devices incorporating the micro- and nano-structures of the present invention may include a biologic glue, an implant, an artificial body part, a tissue engineering and regeneration system, a wound dressing, a synthetic skin, a cell culture matrix, a protein microarray chip, a biosensor, an anastomotic device (e.g., stent), a sleeve, an adhesive film, a scaffold and a coating. Bioadhesive micro- and nano-structures are particularly useful in medical devices configured to provide adhesion or requiring adhesive properties in order to function, for example a biological glue, a wound dressing, a synthetic skin, an adhesive film, or a coating. Use of the bioadhesive micro- and nano-structures of the present invention as a biological glue is particularly beneficial, as there is a significant unmet clinical need for a strong and flexible surgical glue that is highly biocompatible. Introducing adhesive properties to medical devices, such as, but not limited to, an artificial body part, a tissue engineering and regeneration system, a cell culture matrix, a protein microarray chip, a biosensor, an anastomotic device, a sleeve, or a scaffold, which do not generally require inherent adhesion, may still be beneficial. For example, the adhesive properties of said devices may eliminate a need in using additional substances, such as glue, for permanently or releasably attaching the device to the target surface.

In addition to being used as medical devices or being incorporated into medical devices, the micro- and nano-structures of the present invention may be used to provide adhesive coating layers to existing medical devices, or for forming adhesive medical devices, e.g., band-aids.

Sleeves comprising the micro- and nano-structures or compositions described herein can be used, for example, as outside scaffolds for nerves and tendon anastomoses (the surgical joining of two organs).

Adhesive films comprising the micro- and nano-structures or compositions described herein can be used, for example, as wound dressing, substrates for cell culturing and as abdominal wall surgical reinforcement.

Coatings of medical devices comprising the micro- and nano-structures or compositions described herein can be used to render the device biocompatible, having a therapeutic activity, a diagnostic activity, and the like. Other devices include, for example, catheters, aortic aneurysm graft devices, a heart valve, indwelling arterial catheters, indwelling venous catheters, needles, threads, tubes, vascular clips, vascular sheaths and drug delivery ports.

The self-assembled micro- and nano-structures may further be incorporated as cosmetic agents.

As used herein, the phrase “cosmetic agent” refers to topical substances that are utilized for aesthetical purposes. Cosmetic agents in which the micro- and nano-structures and compositions described herein can be beneficially utilized include, for example, agents for firming a defected skin or nail, make ups, gels, lacquers, eye shadows, lip glosses, lipsticks, and the like.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting. As used herein, the singular form “a,” “an,” and “the” include plural references unless the context clearly dictates otherwise. For example, the term “a compound” or “at least one compound” may include a plurality of compounds, including mixtures thereof.

The foregoing examples of the related art and limitations related therewith are intended to be illustrative and not exclusive. Other limitations of the related art will become apparent to those of skill in the art upon a reading of the specification and a study of the figures.

All references cited herein are hereby incorporated by references in their entirety herein.

### EXAMPLES

### Example 1

### Experimental Methods

Nanostructures Self-Assembly

Material—Peptides (Fmoc-DOPA-DOPA, DOPA-DOPA, DOPA-Phe-Phe, Fmoc-DOPA-DOPA-Lys) were purchased from Peptron. Fmoc-DOPA was purchased from Ana Spec. A stock solution of Fmoc-DOPA was prepared by dissolving the building block with ethanol to a final concentration of 100 mg/ml. The stock solution was then diluted into water to the desired concentration (0.5 mg/ml, 0.75 mg/ml, 1 mg/ml, or 2 mg/ml). DOPA-Phe-Phe was prepared by dissolving lyophilized form of the peptide in 1,1,1,3,3,3,-hexafluoro-2-propanol (HFIP), at a concentration of 100 mg/ml or 50 mg/ml. The stock solution was either directly deposited on a cover slip glass slides or diluted into water to a final concentration of 2 mg/ml or 5 mg/ml.

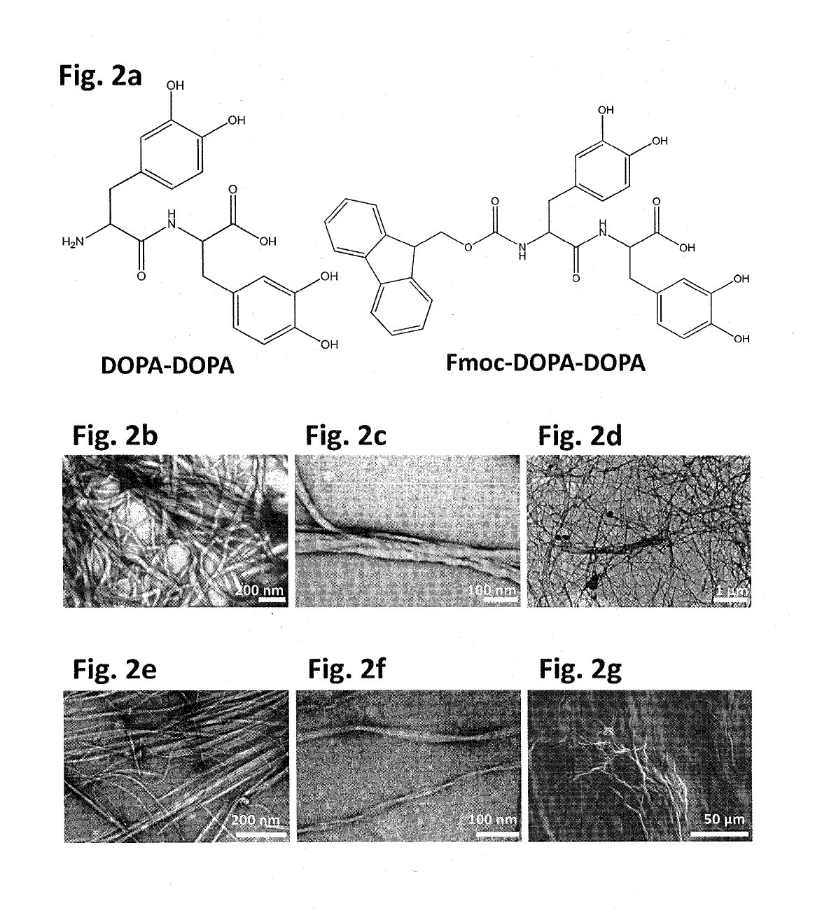

For the formation of the DOPA-DOPA dipeptide asssemblies (FIG. 2A-left panel), lyophilized peptide was dissolved in ethanol to a concentration of 33 mg/mL then diluted with Milli-Q water to a final concentration of 5 mg/mL.

For the formation of the Fmoc-DOPA-DOPA dipeptide assemblies (FIG. 2A-right panel), lyophilized peptide was dissolved in ethanol to a concentration of 100 mg/mL then diluted with Milli-Q water to the desired concentration (2.5 mg/mL, 5 mg/mL or 10 mg/mL).

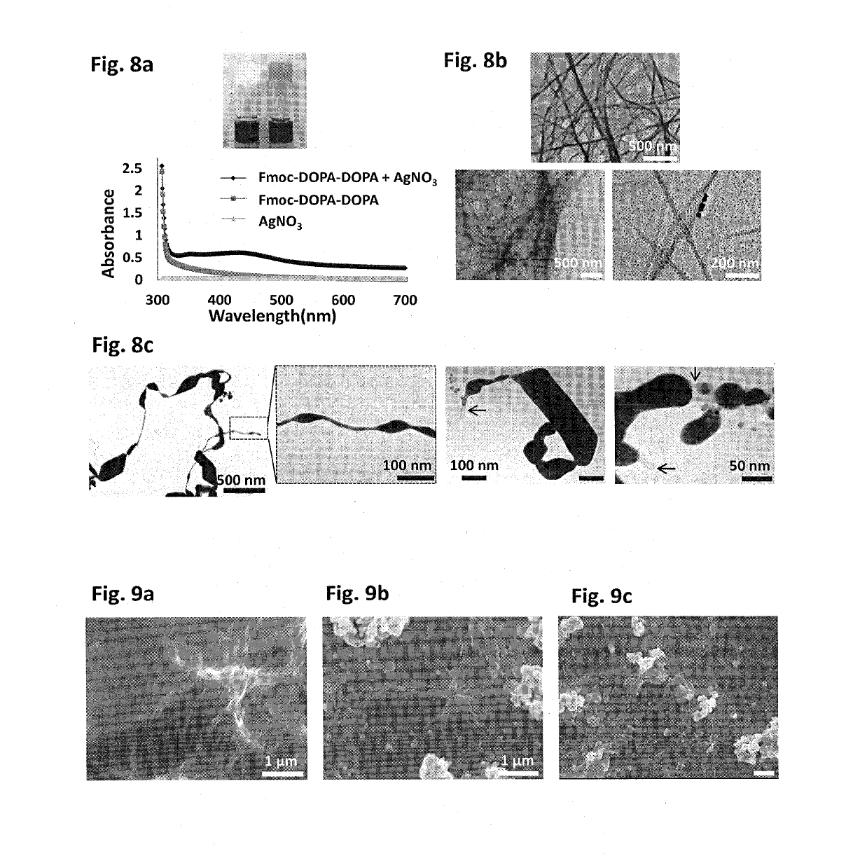

For formation of Fmoc-DOPA-DOPA-Lys assemblies (FIG. 8A), lyophilized peptide was dissolved in either ethanol or dimethyl sulfoxide (DMSO) to a concentration of 100 mg/mL then diluted with Milli-Q water to a final concentration of 12.5 mg/mL.

Asp-DOPA-Asn-Lys-DOPA pentapeptide was synthesized by Peptron Inc. (Daejeon, Korea). To induce the formation of fibrillar assemblies, lyophilized peptide was dissolved in ultra-pure water to concentrations of 100 μM to 15 mM by vortexing followed by bath-sonication for 10 min To avoid pre-aggregation, fresh stock solutions were prepared for each experiment.

Transmission Electron Microscopy (TEM)

TEM analysis was performed by applying 10 μL samples to 400-mesh copper grids covered by carbon-stabilized Formvar film. The samples were allowed to adsorb for 2 min before excess fluid was blotted off. For samples that were negatively stained, 10 μL of 2% uranyl acetate were then deposited on the grid and allowed to adsorb for 2 min before excess fluid was blotted off. TEM micrographs were recorded using JEOL 1200EX electron microscope (Tokyo, Japan) operating at 80 kV.

High Resolution Scanning Electron Microscopy (HR-SEM) for Hydrogel Characterization

HR-SEM analysis was done for Fmoc-DOPA-DOPA hydrogels at different time points after peptide self-assembly. 10 μL samples were dried at room temperature on microscope glass cover slips and coated with chromium. Images were taken using a JEOL JSM 6700F FE-SEM operating at 10 kV.

Environmental Scanning Electron Microscopy E-SEM

E-SEM analysis of 5 mg/mL Fmoc-DOPA-DOPA hydrogel was performed by placing a portion of a pre-prepared hydrogel on a microscope metal stand. Images were taken using Quanta 200 Field Emission Gun (FEG) E-SEM (FEI, Eindhoven, the Netherlands) operating at 10 kV.

### Adhesion Measurements

### Embodiment 1

Atomic Force Microscopy (AFM) was used to assess the adhesion of the formed structures to a silicon oxide tip by employing “force/distance” measurements. This type of measurements allows deducing the attractive forces between the AFM tip and the contacted surface, when this force is represented by the minimum value of a force/distance curve. For AFM analysis, 10 μl aliquot of the peptide suspension was deposited on clean glass slide and dried at room temperature. Force/distance curves were determined at several points using NanoWizardIII of JPK instruments AG.

### Adhesion Measurements

### Embodiment 2

In an alternative embodiment, AFM analysis was performed using an Asylum MFP-1D AFM instrument (Asylum Research, Santa Barbara, Calif., USA). To obtain force data the different peptides were prepared in ethanol: Fmoc-DOPA (1%) Fmoc-DOPA-DOPA (5%), Fmoc-DOPA-DOPA-Lys (12.5 mg/mL), samples were prepared in either DMSO or ethanol (12.5%) these results were compared to measurements performed on bare glass slide.

The AFM measurment was performed by emploting force mapping while simultaneously providing nanoscale topographical and mechanical information about the hydrogel. Force mapping involves generating individual force curves at discrete points on a material, which are then used to calculate stiffness and height values.

After overnight incubation, 10 μL of each sample were deposited on a glass slide and dried at room temperature. Force measurements of the samples were conducted using a SiO₂ colloidal probe (tip velocity 1000 nm/s, compressive force 20 nN). To investigate the glass surface area morphology after peeling, 30 μL of freshly prepared Fmoc-DOPA-DOPA-Lys solutions (12.5 mg/mL, prepared in DMSO or ethanol as described above) were deposited between two microscope glass slides. This construct was incubated overnight at room temperature under 100 mg weight Finally, the upper glass slide was peeled off and the contact area of either slide was imaged using an Ultrasharp AFM probe (NSC21/Ti-Pt, MikroMasch) operated in tapping mode.

Rheological Measurements

Rheological measurements for in situ-formed Fmoc-DOPA-DOPA hydrogel were performed using an AR-G2 rheometer (TA Instruments, New Castle, Del., USA). Time-sweep oscillatory tests in 20 mm parallel plate geometry were conducted at 0.7% strain and 1 Hz frequency on 200 μL of fresh solution (resulting in a gap size of about 0.6 mm), 1 min after its preparation. In order to determine the linear viscoelastic region oscillatory strain (0.01-100%) and frequency sweep (0.01-100 Hz) tests were conducted 45 min after diluting the stock solution with Milli-Q water. All rheology tests were done in triplicates and averaged.

Turbidity Analysis

Turbidity analysis for Fmoc-DOPA-DOPA solutions was conducted using freshly prepared solutions at concentrations of 2.5 and 5 mg/mL. 200 μL aliquots were pipetted into a 96-well plate and absorbance at 405 nm was measured over time, starting 45 s after the preparation of the peptide solution. All measurements were performed using a Biotek Synergy HT plate reader at 25° C.

Metal Deposition on Micro- and Nano-Structures

Fmoc-DOPA-DOPA

Silver reduction assay was performed with pre-prepared Fmoc-DOPA-DOPA hydrogels at a concentration of 5 mg/mL (8.35 mM). 50 μL of 13.2 mM silver nitrate were added to 500 μL hydrogel aliquots by gentle pipetting, resulting in 1.2 mM final concentration of silver nitrate. This solution was incubated at room temperature for several days. At different time points, 10 μL aliquots were taken for TEM analysis and negative staining was not applied. To examine silver reduction using UV-vis spectroscopy, silver nitrate was added to a 5 mg/mL hydrogel pre-prepared as described above and 150 μL aliquots of Fmoc-DOPA-DOPA hydrogel with or without silver nitrate were pipetted into a 96-well UV-Star UV transparent plate (Greiner BioOne, Frickenhausen, Germany) Spectra were collected after 5 days using a Biotek Synergy HT plate reader over the range of 300-700 nm and compared to blank samples of silver nitrate only, or of the peptide only.

DOPA-DOPA

Peptide stock solution was diluted to a final concentration of 5 mg/ml in double distilled water. Then metal salt (AgNO₃) was added to the sample and the samples were analyzed by TEM or HR-SEM. AgNO₃ concentration was determined according to the final catechol concentration in the peptide, with a constant ratio of [AgNO₃]/[Catechol]=0.0723.

Asp-DOPA-Asn-Lys-DOPA

Silver deposition was done by preparing 15 mM peptide solutions and removing residual peptide monomers. This was done by centrifugation at 12,000 RPM for 15 min, discarding the supernatant and resuspention in ultra-pure water. This procedure was repeated once more. Samples for electron microscopy were taken as control and the solution was centrifuged at 12,000 RPM for 15 min and resuspended in an aqueous solution of 2.17 mM AgNO3 for 15 min Finally, the solution was centrifuged at 9000 RPM for 10 min and resuspended in water. Samples for electron microscopy were taken again.

Asp-DOPA-Asn-Lys-DOPA

Silver deposition was done by preparing 15 mM peptide solutions and removing residual peptide monomers. This was done by centrifugation at 12,000 RPM for 15 min, discarding the supernatant and resuspention in ultra-pure water. This procedure was repeated once more. Samples for electron microscopy were taken as control and the solution was centrifuged at 12,000 RPM for 15 min and resuspended in an aqueous solution of 2.17 mM AgNO₃ for 15 min Finally, the solution was centrifuged at 9000 RPM for 10 min and resuspended in water. Samples for electron microscopy were taken again.

Congo Red (CR) Staining

CR staining was performed with 10 μL samples of 6 mM peptide solution. The samples were air-dried on glass microscope slides and staining was performed by the addition of 10 μL solution of 80% ethanol saturated with Congo Red and NaCl. Birefringence was determined using an Olympus SZX-12 Stereoscope (Hamburg, Germany) equipped with a polarizing stage. Fluorescence visualization was performed using Nikon Eclipse 80i epifluorescent microscope (Kanagawa, Japan) equipped with a Y-2E/C filter set (excitation 560/20 nm, emission 630/30 nm). Thioflavin-T (ThT) staining was performed by adding fresh 4 mM ThT solution to an equal volume of 6 mM peptide solution which was incubated for 3 h prior to the addition of ThT. The resultant solution was incubated for 3 h in the dark and 10 μL samples were imaged using LSM 510 Meta confocal microscope (Carl Zeiss, Oberkochen, Germany) at 458 nm excitation and 485 nm emission.

Fourier Transform Infrared (FTIR)

FTIR spectroscopy was performed with 30 μL samples of 6 mM peptide solution deposited onto disposable polyethylene IR sample cards (Sigma-Aldrich, Israel) which were then allowed to dry under vacuum. To achieve hydrogen to deuterium exchange, the peptide deposits were subjected to 2 cycles of resuspension in 30 μL D₂O (99.8%) and drying in vacuum. Transmission infrared spectra were collected using Nexus 470 FTIR spectrometer (Nicolet, Offenbach, Germany) with a deuterated triglycine sulfate (DTGS) detector. Measurements were made using the atmospheric suppression mode, by averaging 64 scans in 2 cm⁻¹ resolution. The amide I′ region was deconvoluted by subtracting a baseline of ultra-pure water that was deposited on a polyethylene sample card and subjected to two cycles of resuspension in D₂O as described above. Subtraction was performed using the OMNIC software (Nicolet). Smoothing, second derivative calculation and curve-fitting were then performed using the Peakfit software version 4.12 (SYSTAT, Richmond, Calif.). For transmittance plots, 13-data-point savitzky-golay smoothing was applied to the raw spectra using the Omnic software.

Circular Dichroism (CD)

CD spectroscopy was performed by dilution of fresh 6 mM peptide solution in ultra-pure water to a concentration of 60 μM. CD spectra were collected with a Chirascan spectrometer (Applied Photophysics, Leatherhead, UK) fitted with a Peltier temperature controller set to 25° C., using a capped rectangular quartz cuvette with an optical path length of 0.1 cm. Absorbance was kept under two units during all measurements. Data acquisition was performed in steps of 1 nm at a wavelength range from 190-260 nm with a spectral bandwidth of 1.0 nm and an averaging time of 3 s. The spectrum of each sample was collected three times and a control spectrum of ultra-pure water was collected twice. Spectra were corrected in baseline with ultra-pure water as the blank. Data processing was done using Pro-Data Viewer software (Applied Photophysics, Leatherhead, UK); processing and normalization to mean residue ellipticity (MRE). To verify the assayed solution contained characteristic assemblies, a 10 μL sample of the cuvette content was examined by TEM as described above.

Thermal perturbation was performed using freshly made 15 mM peptide solution diluted to a concentration of 120 μM. CD spectra were collected as described above except that the spectra were obtained throughout temperature variation done in a stepwise fashion up and then down. The investigated temperatures ranged over 25° C.-90° C. (in the following steps: 25° C., 37° C., 50° C., 70° C., 90° C., 70° C., 50° C., 37° C., 25° C.). The sample was allowed to equilibrate for 10 min and the temperature was monitored by a thermocouple in the cuvette holder block. At the end of the measurments, the cuvette content was sampled for TEM and FTIR analysis as described above. As control, TEM and FTIR samples were taken from an aliquot of the same solution which was not subjected to temperature variations.

### Results

### Example 2

### Fmoc-DOPA and Fmoc-DOPA-DOPA

Morphology Analysis

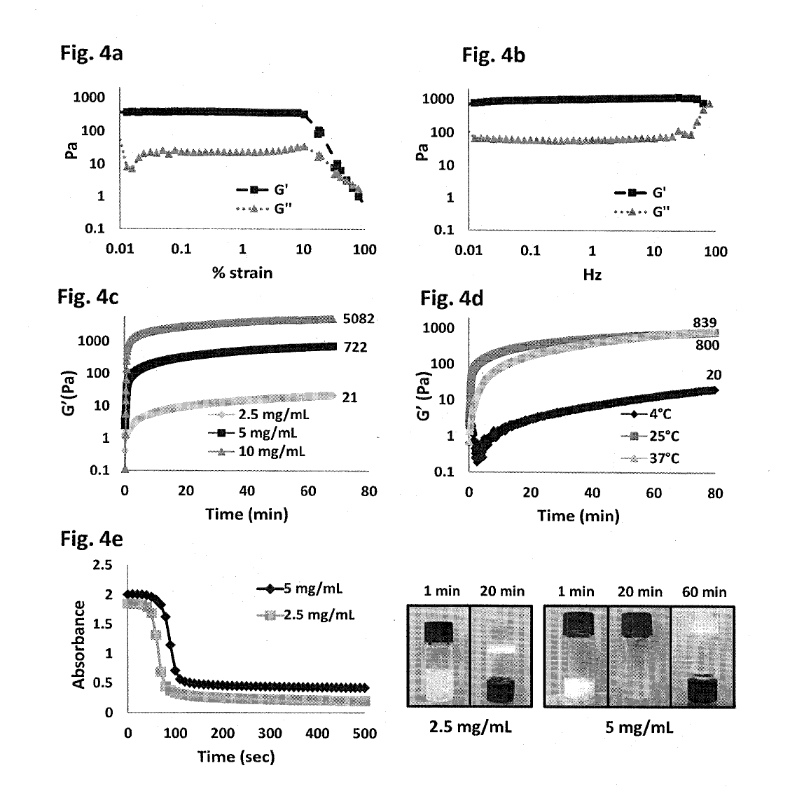

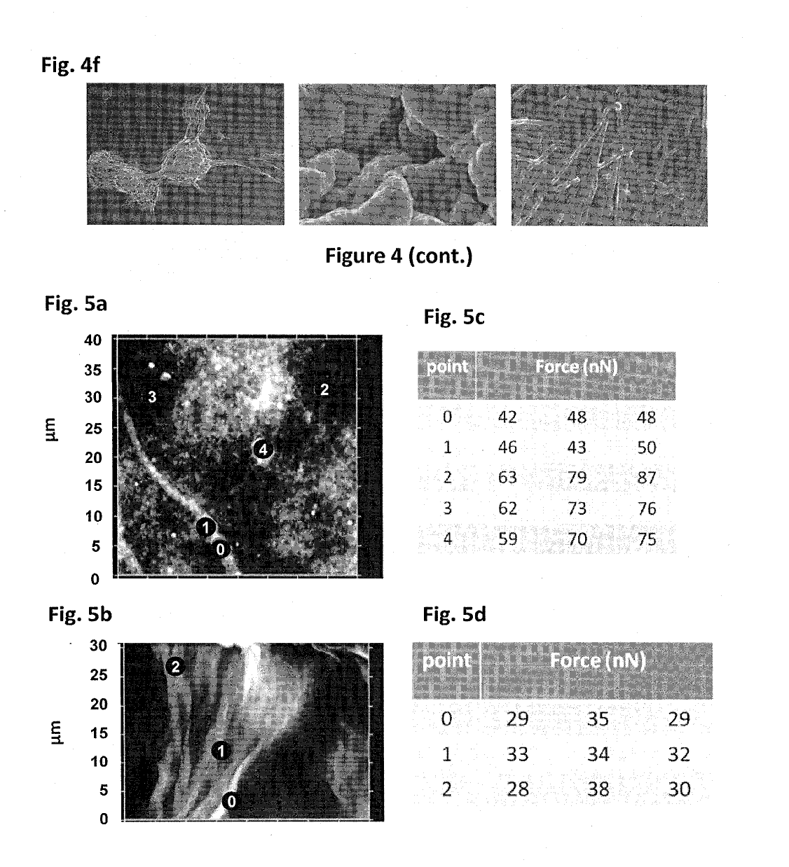

DOPA-DOPA and Fmoc-DOPA-DOPA peptides were examined under different conditions and were found to self-assemble into ordered nanostructures in tihe presence of ethanol and water (FIG. 2B-G). Mactroscopically, the Fmoc-DOPA-DOPA peptide formed a self-supporting hydrogel (FIG. 4). To gain a better insight into the molecular organization and morphology of the formed structures, electron microscopy was employed. Transmission electron microscopy (TEM) analysis of both peptides revealed the formation of a tangled fibrous network composed of flexible, elongated fibrillar structures. The existence of twisted multistrand fibers alongside single fibrils was observed. The DOPA-DOPA dipeptide assembled into fibers with a cross section ranging from 20 to 50 nm (FIG. 2B, 2C) while Fmoc-DOPA-DOPA formed narrower fibers, varying in width from approximately 4 to 30 nm (FIG. 2D, 2E, 2F).

To characterize the morphology of the Fmoc-DOPA-DOPA hydrogel under humid conditions, environmental scanning electron microscopy (E-SEM) was carried out. Upon gradual dehydration of the sample, a network of supramolecular substructures was observed (FIG. 2G).

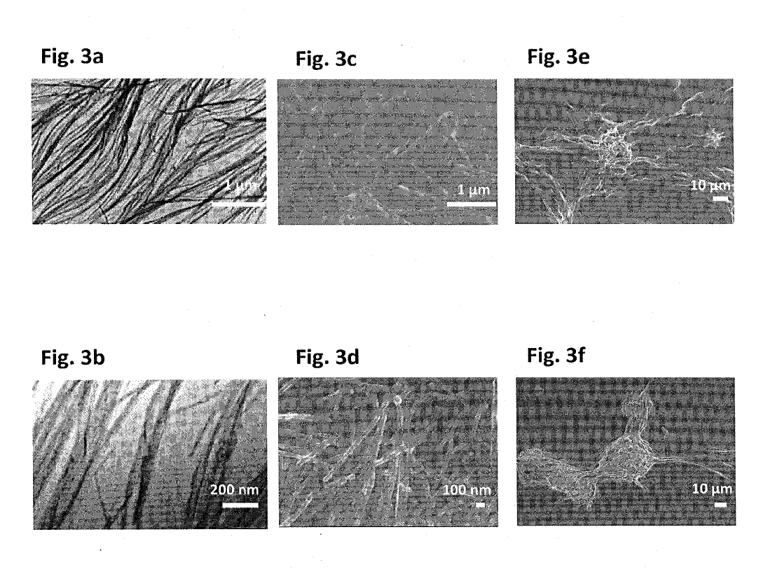

In another experiment, norphology characterization of the hydrogel revealed fibrous network with fibril diameters ranging from 20 to 100 nm (FIG. 3A-3B). The fibrils forming the network are flexible with branching characteristics. HR-SEM samples taken minutes or hours after the assembly process (FIG. 3C-3F) may explain the changes in the optical properties of the sample over time. At first, large aggregates (diameter of 20-50 μm) were observed, whereas after several hours ordered structures with smaller diameter (20-100 nm) were seen. This observation fits a theory suggesting that the duration of the optical transition in Fmoc-DOPA-DOPA to the time required for the initial organization of the molecules to undergo a physical restructuring. This restructuring, from irregular aggregates with dimensions in the range of the visible wavelength to ordered structures with final diameters smaller than the visible light wavelength, causes the changes in the optical characteristics of the solution. In contrast to the gelation process, the decrease in turbidity was not temperature dependent. This indicates that additional parameters, other than temperature, affect the self-assembly process of the hydrogel. The assumption is that spontaneous oxidation that occurs over time has a key-role in stabilizing the formed structures. Oxidation of DOPA to DOPA-quinone or DOPA-semiquinone can lead to cross-linking, giving rise to solidification of the hydrogel.

Rheological Analysis

The hydrogel formed by the low molecular weight (LMW) Fmoc-DOPA-DOPA peptide was further characterized. The viscoelastic properties of the gel were assessed using rheological measurements. Oscillatory strain (0.01-100%) and frequency sweep (0.01-100 Hz) tests were conducted to determine the linear viscoelastic regime (FIG. 4A-4B). These tests revealed that at the linear region, the storage modulus (G′) of the hydrogel is more than one order of magnitude larger than the loss modulus (G″), a rheological behavior that is characteristic of elastic hydrogels. As shown in FIG. 4C, the plateau storage modulus of the hydrogel was found to be modulated with a high dynamic range of ˜20 Pa to ˜5 kPa, corresponding to the final concentration of the peptide. Furthermore, the gelation kinetics was found to be temperature dependent (FIG. 4D), as gelation was highly decelerated at 4° C. compared to 25° C. or 37° C. At higher temperatures (25° C. or 37° C.), the storage moduli of the hydrogels were approximately 40 fold higher than the storage modulus of hydrogels formed at 4° C. The gelation process of Fmoc-DOPA-DOPA was also accompanied by a change in the optical properties of the sample, transforming from a turbid viscous solution to a semi-transparent hydrogel (FIG. 4E-left panel). When the 2.5 mg/mL sample was observed macroscopically, the solution cleared within minutes, corresponding to the formation of the hydrogel (FIG. 4E-center panel). In contrast, in the case of the 5 mg/mL, although the solution cleared within minutes gelation occurred after longer periods of time (FIG. 4E-right panel). High-resolution SEM (HR-SEM) analysis of the 5 mg/mL samples taken minutes or hours after the assembly process (FIG. 4F) indicated that large aggregates are present at the initial stage when the solution is turbid, whereas after several hours, ordered structures with much smaller diameter appear, corresponding to a considerably clearer solution. This observation is also in line with a previous hypothesis put forward by the present inventors linking the optical transition from turbid to transparent in Fmoc-protected hydrogel preparation to the ultrastructural organization over time [28]. This restructuring, from irregular aggregates with dimensions similar to or higher than the wavelengths in the visible spectrum to ordered structures with final diameters much lower than these wavelengths, results in the change of the scattering properties of the solution.

Adhesion Measurements

The adhesion of Fmoc-DOPA-DOPA adhesion to silicon oxide was intially estimated as described in Example 1 (Adhesion measurements-Embodiment 1) where a peptide solution at two different concentrations (2 mg/ml and 5 mg/ml) were deposited onto a glass slide and imaged with the AFM (FIGS. 5A and 5B, respectively). The adhesion force was found for 2 mg/ml (5 locations) and 5 mg/ml (3 locations) by measuring the interaction of the AFM tip and the surface. The adhesion of the AFM tip to the Fmoc-DOPA-DOPA sample ranged between 28 to 87 nN as summarized in the tables (FIG. 5C-5D).

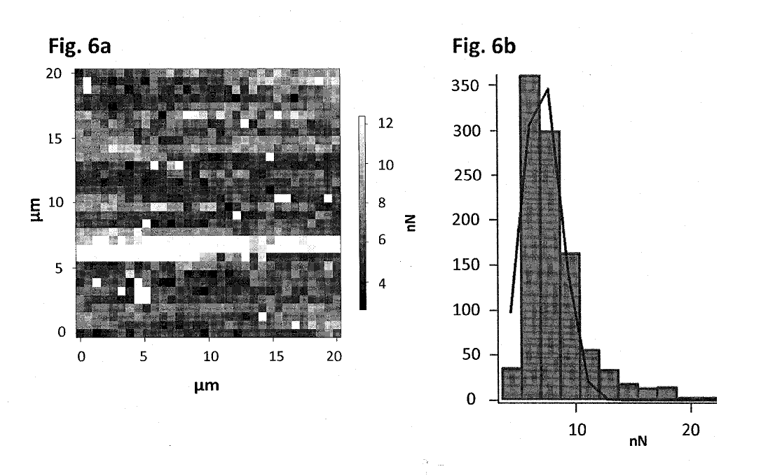

The adhesion of the peptide to silicon oxide was measured as described in Example 1 (Adhesion measurements-Embodiment 2). AFM measurements were initially performed on a bare glass slides and a (20 μm×20 μm) force map (FIG. 6A) was generated. The adhesion force map on the glass slide (FIG. 6B) resulted in an adhesion histogram in which the mean force is 7±1.2 nN.

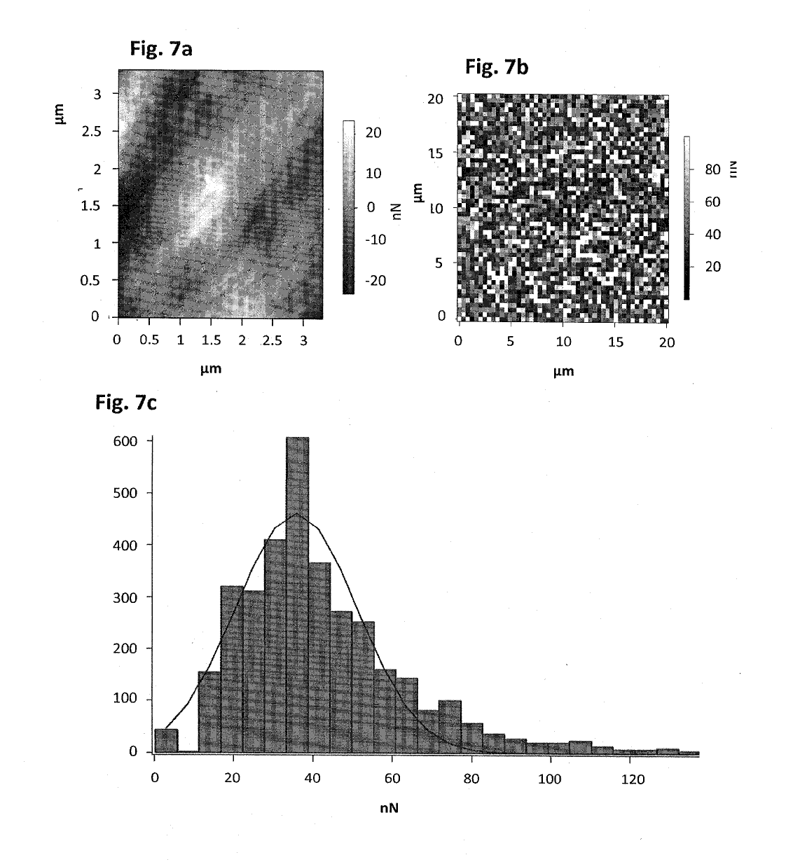

Fmoc-DOPA-DOPA peptide (5 mg/ml) was deposited onto a glass slide and imaged with the AFM in tapping mode (with a ultra sharp MicroMasch AFM probe), with a scan size of 3×3 μm² (FIG. 7A). This revealed the presence of bulk fibrous structures. The adhesion force of the AFM tip (SiO₂ colloidal probe) to the Fmoc-DOPA-DOPA peptide was performed by generating a 20×20 μm² force map (FIG. 7B) to the AFM. From the adhesion histogram (FIG. 7C) the mean force was calculated to be 36±11 nN. The tip velocity was 1000 nm/s and the compressive force was 20 nN.

Redox Properties

The catechol group of the DOPA moiety has redox properties that enable the spontaneous reduction of metal cations into neutral metal atoms resulting in metal nanoparticles. The Fmoc-DOPA-DOPA based hydrogel was tested for having the inherent property to spontaneously form silver particles from silver nitrate. During the assembly process of Fmoc-DOPA-DOPA, the catechol functional group could either be directed towards the inner core of the assembled structure or towards the solution. It was hypothesized that DOPA groups that are not facing towards the hydrophobic core of the structures will be able to react with the silver salt to form silver particles that can be easily detected using electron microscopy. Moreover, the formation of Ag is accompanied by a color change resulting in an absorbance peak centered at ˜400 nm that can be easily be observed by the naked eye.

The addition of silver nitrate solution to a pre-prepared Fmoc-DOPA-DOPA hydrogel led to a change in the hydrogel color from semi-transparent to dark brown over a period of several hours to a few days (FIG. 8A). UV-vis spectra taken 5 days after the silver nitrate was added to the hydrogel revealed an increase in the absorbance above 300 nm. The broadband increase in the absorption in this area could be due to scattering by silver nanoparticles and DOPA oxidation.

The ionic silver reduction process was further monitored by TEM. A slow, gradual transition from the formation of local silver nanoparticles nuclei to the formation of a continuous silver layer was observed (FIG. 8B). Distinct formation of silver nanoparticles was observed after short incubation or at a low concentration of the peptide. However, after a longer incubation at a high peptide concentration, the seamless coating of the peptide assemblies was clearly observed (FIG. 8C). Such uniform and continuous coating is unique and is most likely the result of both the slow reduction kinetics, as discussed above, and the high density of catechol groups presented by the assemblies.

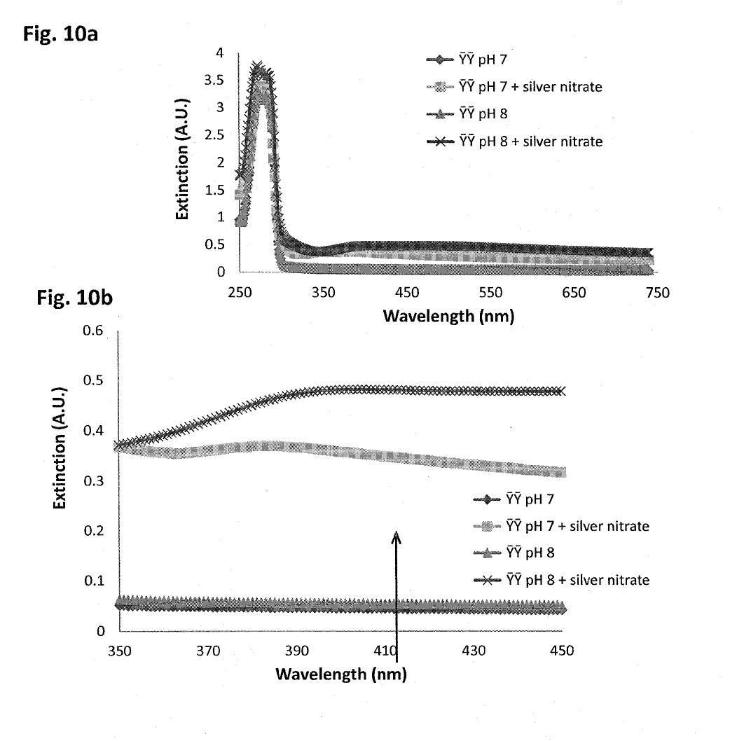

The reaction of DOPA-DOPA dipeptide (FIG. 9A) with silver nitrate had resulted in the formation of silver particles (FIG. 9B-9C). This reduction of the silver-nitrate (AgNO₃) can also be seen in the appearance of an absorbance peak (FIG. 10A) at 390 nm (reaction at pH=7) and at 400 nm (reaction at pH=8). A zoom-in of the absorbance peaks (FIG. 10B) further reveals the differences in absorption values in the presence and absence of silver nitrate.

### Example 3

### Fmoc-DOPA

Morphology Analysis

AFM and light microscpy were both employed to study the self-assembly properties of Fmoc-DOPA peptide. It was found that under different conditions ordered spheres-like structures were formed with diameters ranging between dozens of nm to microns. Measurements were conducted on samples of Fmoc-DOPA at different concentrations (2 or 5 mg/ml), pH (5.5 or 8.7) and in the absence or presence of Fe⁺³. The hypothesis is that both basic pH and Fe⁺³ ions may contribute to the adhesion of Fmoc-DOPA.

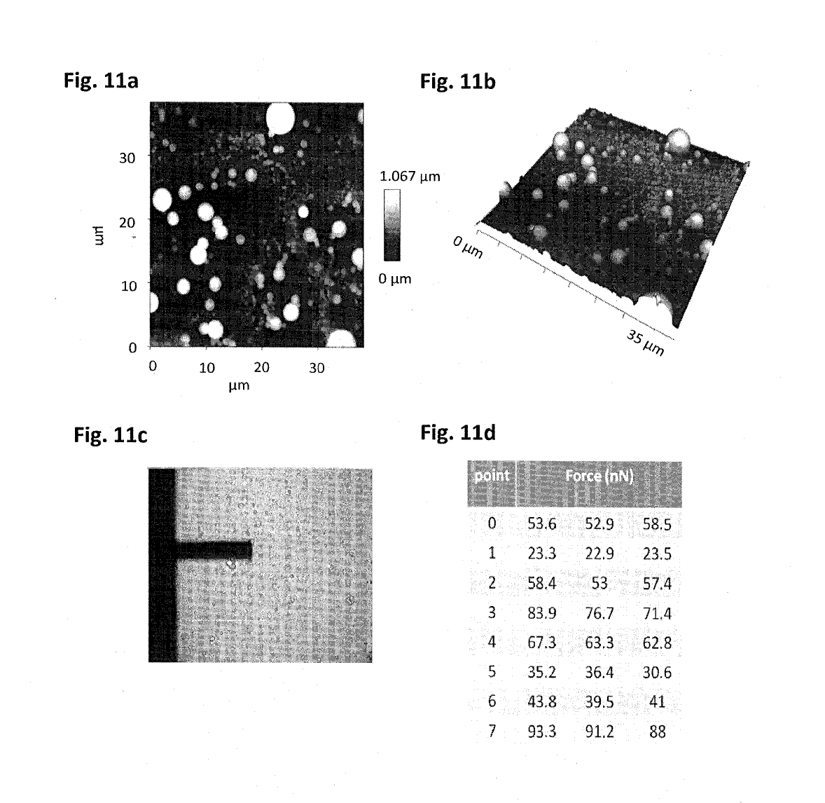

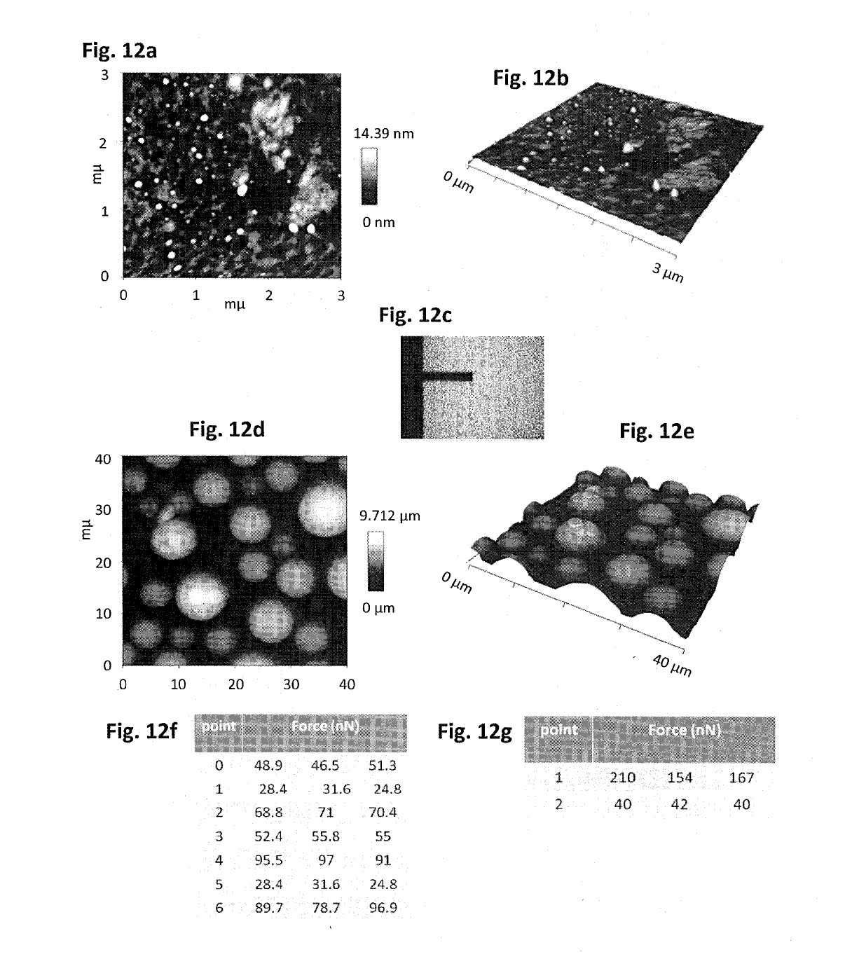

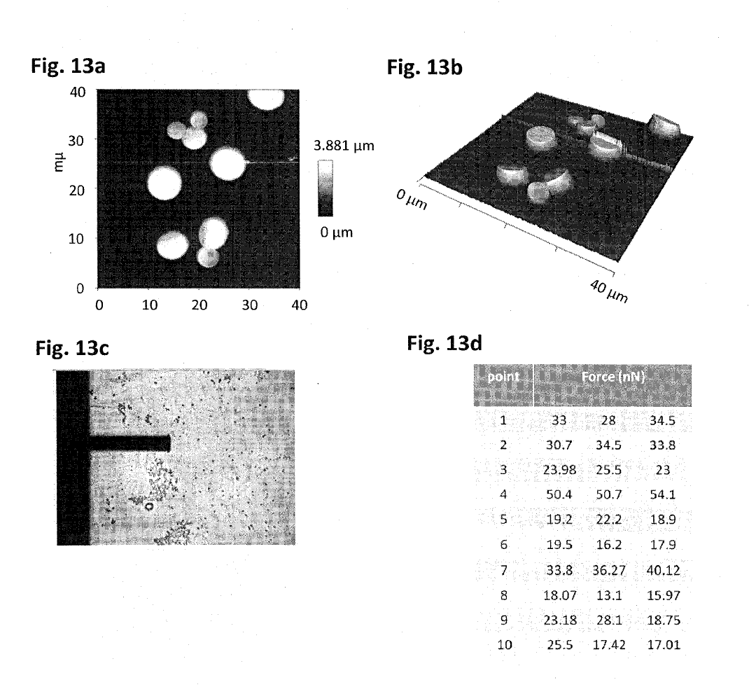

In general all experiments were performed with water (pH 5.5). In some cases, the water medium was pre-adjusted to the specific pH mentioned by the addition of diluted NaoH solution. Due to the non-buffered conditions, after addition of the acidic peptides, the over all pH of the system was acidic. Examination of 5 mg/ml Fmoc-DOPA at acidic pH (˜5.5) showed sparse spheres at varied sizes (FIG. 11A-11C). Measurements of 5 mg/ml Fmoc-DOPA revealed a dense layer of spheres with variying sizes, having diameters ranging between dozens of nano-meters to microns (FIG. 12A-12E). A sample of 5 mg/ml Fmoc-DOPA at with the presence of Fe⁺³ also displayed a dense layer of spheres at varied sizes. Fmoc-DOPA at lower concentration (2 mg/ml) also exhibited diversity in structures sizes, with a population composed of large truncated spheres and small spheres (FIG. 13A-13C).

Adhesion Measurements

Several force-distance curves have been taken, as described in Example 1 (Adhesion measurements-Embodiment 1), to measure the adhesion of the Fmoc-DOPA peptide under the various conditions described above. The results of the adhesion measurements for the 5 mg/ml Fmoc-DOPA at acidic pH (5.5) sample are summarized in the table in FIG. 11D. The adhesion of the AFM tip to the Fmoc-DOPA peptide was found to range between 23 nN to 93 nN. Adhesion measurements for the 5 mg/ml Fmoc-DOPA sample ranged between 24 to 97 nN (table in FIG. 12F). Adhesion measurements for the sample with the presence of Fe⁺³ ranged between 40 nN to 210 nN (Table in FIG. 12G). Adhesion measurements for 2 mg/ml Fmoc-DOPA sample resulted in values ranging between 13 nN to 50 nN (table in FIG. 13D).

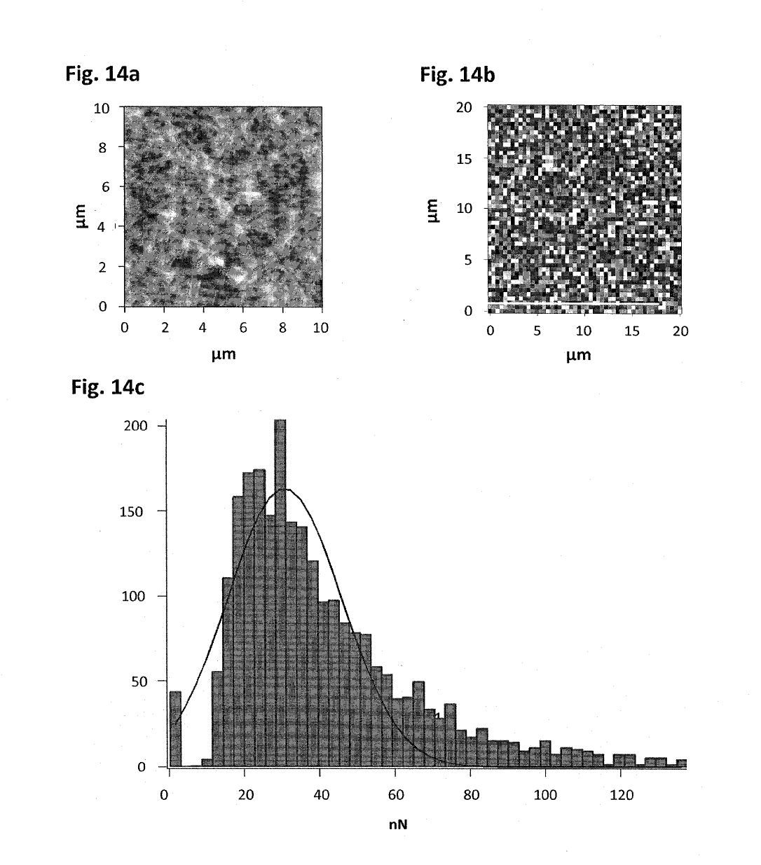

A 10×10 μm² topography (FIG. 14A) and a 20×20 μm² force map (FIG. 14B) images of the deposited Fmoc-DOPA (1 mg/ml) were taken (Adhesion measurements-Embodiment 2) followed by the construction of an adhesion-force histogram (FIG. 14C). It was found that the mean adhesion force was 31±10.6 nN, a similar value to that found for the Fmoc-DOPA-DOPA peptide using the same measurement. The tip velocity was 1000 nm/s and the compressive force was 20 nN.

### Example 4

### Fmoc-DOPA-DOPA-Lys

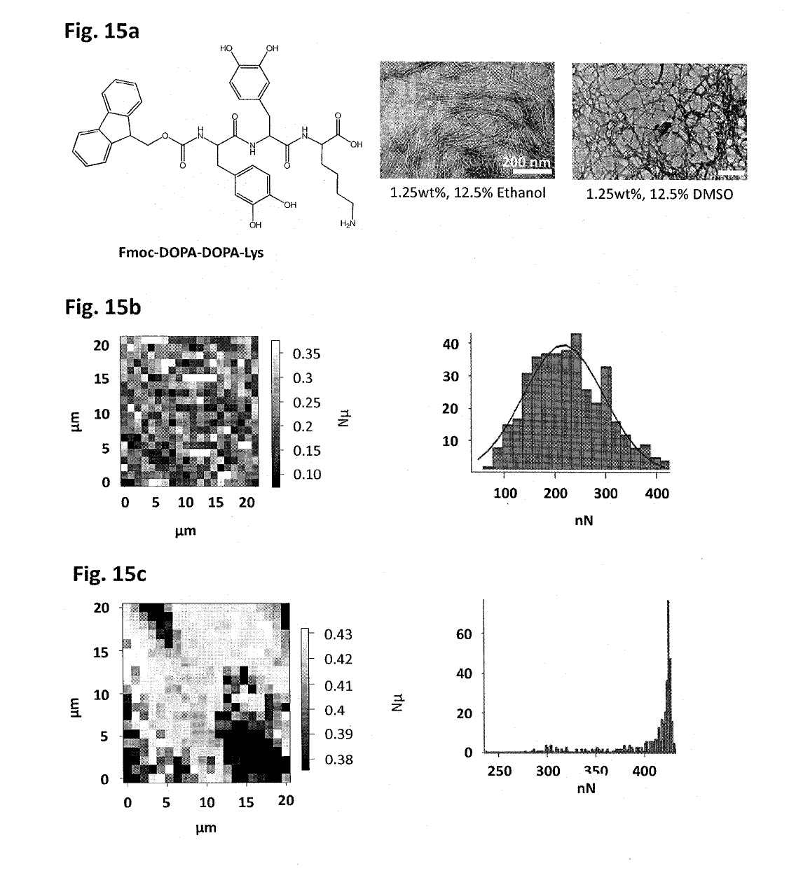

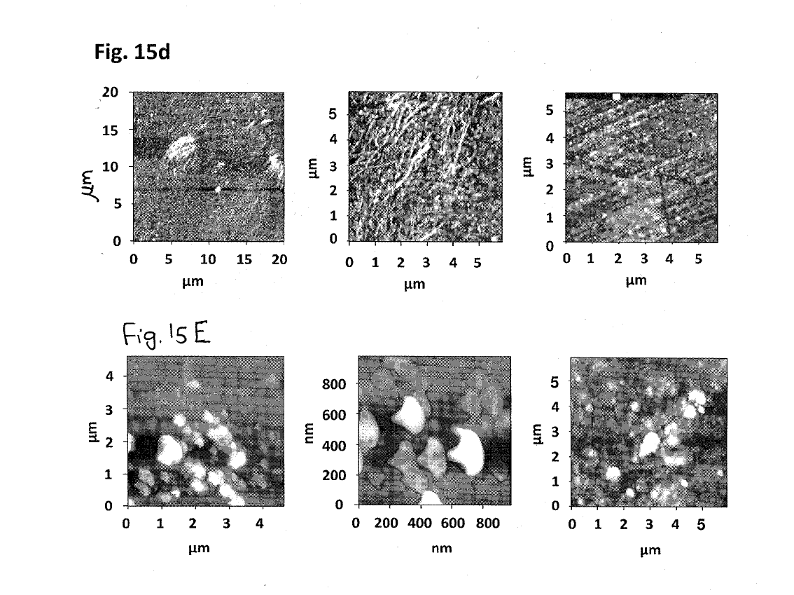

It was hypothesized that the incorporation of a lysine residue into the DOPA-containing peptide assemblies would contribute to cohesion and thus indirectly improve its adhesion. Moreover, lysine residues may also contribute to adhesion via ionic bonding to negatively charged surfaces. Therefore, the Fmoc-DOPA-DOPA-Lys (FIG. 15A-left panel) protected tripeptide was designed and tested.

Morphology Analysis

Upon examination of the protected tripeptide under the conditions applied to the two peptides studied initially (DOPA-DOPA and Fmoc-DOPA-DOPA), the Fmoc-DOPA-DOPA-Lys peptide was also found to self-assemble into well-ordered fibrillar structures. However, in contrast to the fibers formed by the former, the fibers assembled by the Fmoc-DOPA-DOPA-Lys were narrower, with an approximated width of less than 10 nm. Moreover, the fine fibers were only formed by dissolving the peptide to higher concentrations of (1.25 wt % versus 0.5 wt %) as can be seen in the center panel of FIG. 15A. Fmoc-DOPA-DOPA-Lys was also found to self-assemble into ordered nanostructures in the presence of dimethyl sulfoxide (DMSO) and water (FIG. 15A-right panel), forming assemblies that also displayed a high degree of ultrastructural similarity to the Fmoc-DOPA-DOPA structures.

Adhesion Measurements

To quantify the adhesive forces of the tripeptide sample to the glass surfaces, AFM measurements were taken, as described in Example 1 (Adhesion measurements-Embodiment 2). Specifically, the adhesion of the structures to a silicon oxide (SiO₂) colloidal probe was assessed by employing force-distance measurements. In comparison to the very low adhesion of the AFM probe to bare glass, a glass surface covered with Fmoc-DOPA-DOPA-Lys tripeptide assemblies displayed significant adhesive forces. Whereas the adhesion of the tip to bare glass was less than 10 nN (FIG. 6B), it was found that the adhesion of the Fmoc-DOPA-DOPA-Lys to the glass was calculated to be more than 214 nN when prepared in ethanol (FIG. 15B) and 300 nN (FIG. 15C) when prepared in DMSO.

Macroscopically, it was observed that this protected tripeptide forms viscoelastic glue capable of adhering two glass slides. Under both preparation conditions (i.e. ethanol and DMSO), the sample displayed macroscopic adhesive properties, capable of gluing together two glass slides. It should be noted that this gluing phenomenon, in the presence of DMSO, showed recovery behavior: after peeling the glass of from each other they were able to be re-joined. Interestingly, this was not the case when ethanol was used for the preparation of Fmoc-DOPA-DOPA-Lys solutions. When this procedure was repeated with an ethanol-prepared tripeptide sample, the sample lost its adhesive properties after peel forces were applied.

To understand the basis for the recovery differences between the DMSO and ethanol tripeptide samples, after the two glass slides were glued together we peeled off the glass slide and examined the exposed area. AFM analysis of Fmoc-DOPA-DOPA-Lys in ethanol after peeling (FIG. 15D) exhibited unidirectional fine fibrous structures. In contrast, AFM analysis of Fmoc-DOPA-DOPA-Lys in DMSO after peeling (FIG. 15E) revealed the presence of twisted spheres that were unidirectionally retracted.

### Example 5

### DOPA-Phe-Phe

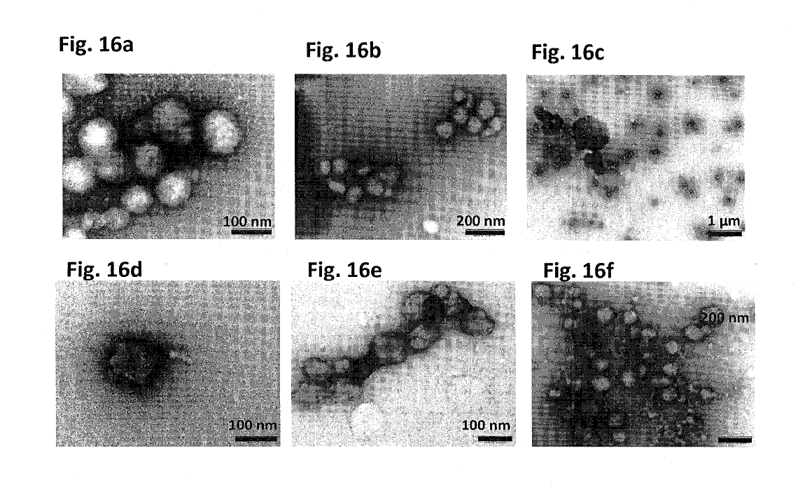

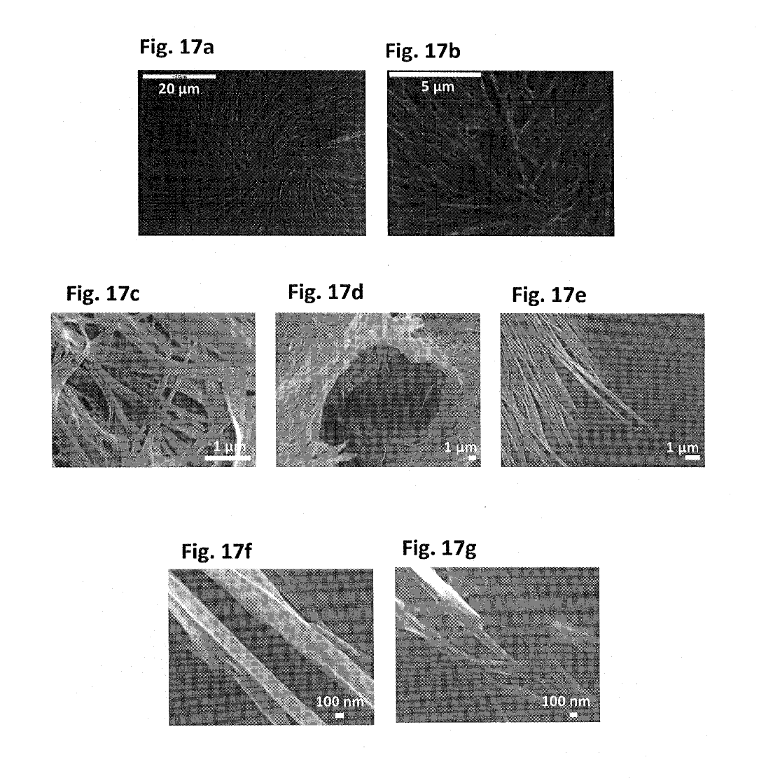

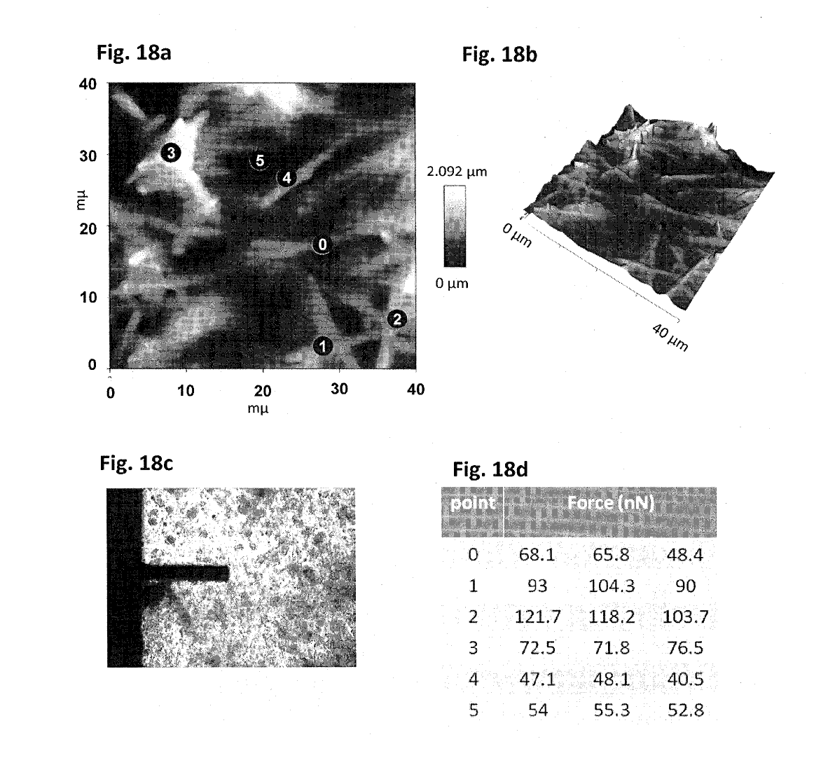

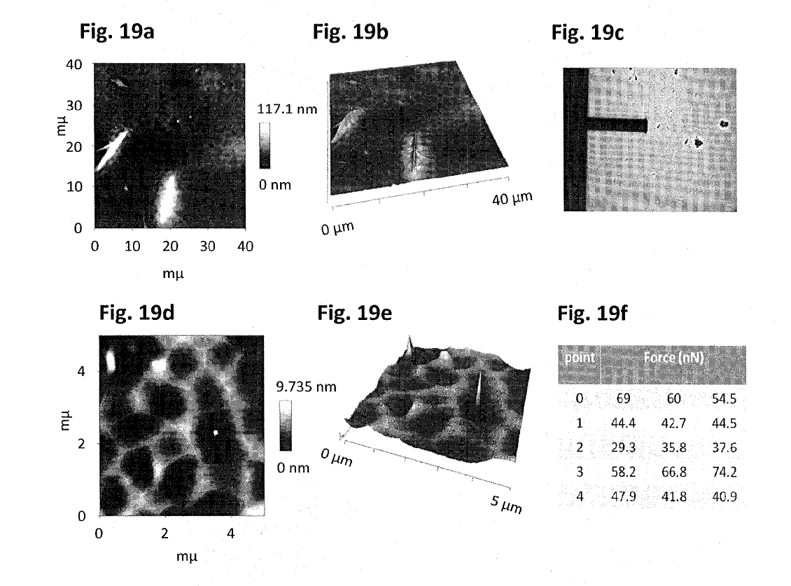

The self-assembly ability of DOPA-Phe-Phe was examined Examination of the DOPA-Phe-Phe peptide revealed that this peptide assembles into sphere-like particles, with diameters ranging between 30 to 100 nm, when it is dissolved in HFIP (100 mg/ml) and then diluted in water to a final concentration of 2 or 5 mg/ml (FIG. 16A-16F). In addition, DOPA-Phe-Phe (50-100 mg/ml), in the presence of 100% HFIP, was found to form highly dense horizontal aligned ribbon-like structures with diameters ranging between 30 to 100 nm. These structures were found to be filamentous, branched and flexible (FIGS. 17A-17G and FIGS. 18A-18C). 100 mg/ml DOPA-Phe-Phe formed dense tube-like ordered structures Examination of individual tube-like structures formed by 50 mg/ml DOPA-Phe-Phe revealed, at the 3D imaging, wall-like structures with cratered surface (FIGS. 19A-19E). The spatial arrangement of the peptides is probably due to the rapid evaporation of HFIP.

Adhesion Measurements

100 mg/ml DOPA-Phe-Phe sample adhesion was found to be in the range from 40 nN to 121 nN (FIG. 18D). Adhesion measurements of 50 mg/ml DOPA-Phe-Phe sample were between 29 to 74 nN (FIG. 19F).

In summary, the aforementioned examples show that the substitution of phenylalanine with DOPA in the known self-assembling peptide motif FF yields novel self-assembling peptides that are able to form ordered supramolecular structures substantially decorated with catechol functional groups. Due to the intrinsic properties of the catechol group, the obtained supramolecular structures can be used as new multifunctional platforms for various technological applications. Upon the incorporation of additional lysine residue containing 6-amine, significant adhesion was obtained, possibly due to electrostatic interactions between the protonated amine and negatively charged oxide surface. The remarkable seamless silver deposition reflects the tendency of the dense catechol array to facilitate coating rather than adhesion. The properties of this deposition are unique, as compared to any known electroless metal coating of biological or polymer nano-assemblies and should prove very useful in the templating of inorganic materials on organic surfaces at the nano-scale for various applications, e.g., formation of anti-bacterial hydrogels, among others.

### Example 6

### Pentapeptides DOPA-Containing Fibrillar Assemblies

Amyloid fibrils self-assemble through molecular recognition facilitated by short amino acid sequences found in amyloidogenic proteins or polypeptides, which were identified as minimal amyloidogenic recognition modules. These modules can serve as initiators and facilitators of aggregation and vary between amyloidogenic proteins and polypeptides. By employing a reductionist approach, in vitro studies utilizing short synthetic peptides as model systems led to the discovery of minimal recognition modules in numerous amyloidogenic proteins and polypeptides. One such module was identified in human calcitonin (hCT), a 32-residue polypeptide hormone which plays a role in calcium-phosphate homeostasis. hCT can form amyloid fibrils in vivo and the fibrils were implicated in the pathogenesis of medullary thyroid carcinoma. The sequence Asp-Phe-Asn-Lys-Phe (SEQ ID. No. 2). was previously identified as the minimal amyloidogenic recognition module of hCT [15]. This pentapeptide, spanning residues 15-19 of hCT, forms amyloid fibrils in vitro at neutral pH in aqueous solutions with remarkable similarity to the fibrils formed by the full-length hCT.

Self-assembly of amyloid-like structures has been a subject of much interest in nanobiotechnology. Due to their ability to self-assemble into ordered nanostructures that may also be chemically and biologically functionalized, amyloidogenic peptides are regarded as promising building blocks for various nanobiotechnological applications.

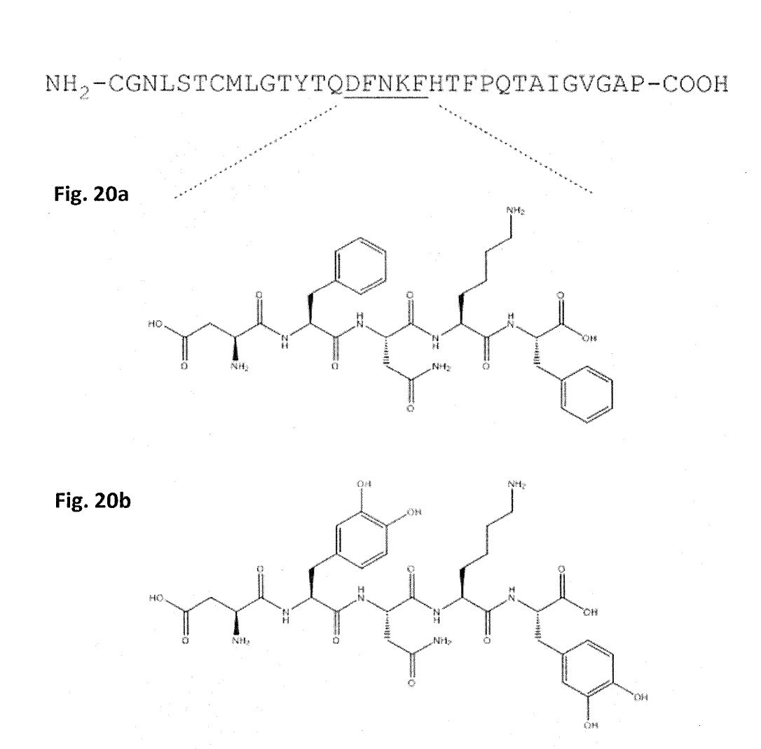

As demonstrated herein, a short synthetic peptide, the minimal amyloidogenic recognition module of hCT (SEQ ID ID No. 1) (FIG. 20A), was designed to contain two DOPA moieties, substituting the phenylalanine residues, resulting in a unique building block, the Asp-DOPA-Asn-Lys-DOPA pentapeptide (FIG. 20B). This pentapeptide retains the ability to spontaneously self-assemble in vitro into amyloid-like fibrillar assemblies in simple aqueous solutions. The obtained assemblies displayed structural properties characteristic of amyloids as well as characteristics of DOPA-containing polypeptides. Functional assessment of the assemblies suggested redox activity and demonstrated the applicative potential of this novel nanobiomaterial.

### Morphology Characterization

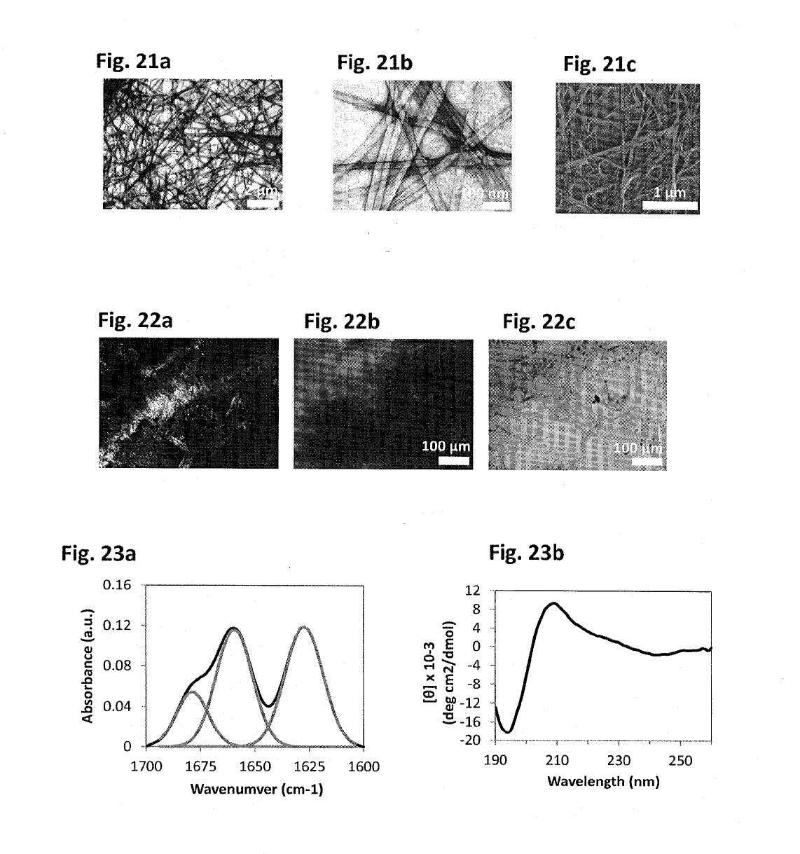

Lyophilized Asp-DOPA-Asn-Lys-DOPA peptide was dissolved in aqueous solutions followed by the application of bath-sonication for 10 min Concentrations of 100 μM to 15 mM were tested and a viscous turbid solution was obtained in all cases. Samples were examined by TEM (FIGS. 21A-21B) and E-SEM (FIG. 21C). A network of fibrillar assemblies was detected and the observed fibrillar assemblies were mostly linear, unbranched and extending to the length of micrometers. The width of individual fibrillar assemblies varied from 15 to 85 nm and lateral bundling of the assemblies was observed. Such morphological features are common to amyloids and amyloid-like structures.

### Secondary Structure Analysis

A hallmark of the amyloid cross-β structure is apple-green birefringence of the dye Congo Red (CR) under polarized light when bound to amyloid fibrils. This can be supported by CR fluorescence, which gives red-orange emission (616 nm) upon green excitation (510-560 nm). When Asp-DOPA-Asn-Lys-DOPA samples were dried and stained with CR, apple-green birefringence (FIG. 22A) and red-orange fluorescence (FIG. 22B) were observed while virtually none were observed in control samples of CR only or of the peptide without staining. FIG. 22C represents brightfield image corresponding to the fluorescence microscopy micrograph (scale bars represent 100 μm).

Further analysis was performed using transmission Fourier transform infrared (FTIR) spectroscopy. Samples of 6 mM Asp-DOPA-Asn-Lys-DOPA solutions were dried, subjected to hydrogen-to-deuterium exchange and analyzed. The second derivative of the amide I′ region (1600-1700 cm-1) was curve-fitted and component bands were assigned to secondary structure elements (FIG. 23A). Distinct peaks were found at approximately 1627 and 1679 cm-1, which are attributable to the presence of β-sheet structures. The combined presence of a low component around 1630 cm-1 and a weaker, high component around 1680 cm-1 is consistent with an antiparallel β-sheet structure. Indeed, an antiparallel β-sheet structure, albeit with slightly different peak positions, was previously reported for fibrils formed by the unmodified hCT minimal recognition module. A third peak, at 1659 cm-1, can be attributed to α-helical structures. It was reported that such structures appear in solutions of the unmodified hCT minimal recognition module in a concentration-dependent manner.

To examine the secondary structure of Asp-DOPA-Asn-Lys-DOPA in solution, freshly made 6 mM solutions were diluted to a final concentration of 0.15 mM and circular dichroism (CD) spectroscopy in the far-UV region was performed at 25° C. Weak negative ellipticity was observed at approximately 235-250 nm, followed by a weak positive shoulder around 230 nm, a positive maximum at 209 nm and a negative maximum at 194 nm (FIG. 23B). A remarkably similar CD spectrum has been previously reported for the highly-polymerized poly(L-Lys-L-Lys-L-Lys-L-DOPA) sequential polypeptide in water at 25° C. A negative maximum near 195 nm indicated that the sequential polypeptide adopted a random coil conformation under these conditions, whereas the positive ellipticity around 220 nm was attributed to transitions of the catechol side-chain and not to secondary structure elements. The CD spectrum of Asp-DOPA-Asn-Lys-DOPA therefore suggests that the peptide adopts a random coil conformation in solution. This result is surprising on several counts. First, a characteristic random coil band in the Amide I′ region was not detected by FTIR spectroscopy. Second, TEM examination of solution samples taken from the CD cuvette at the end of the measurement contained ample amyloid-like fibrillar assemblies as described above (data not shown); since no additional CD bands were detected and since the ultrastructure was not lost, the CD measurement seems to represent the secondary structure of the peptide while being part of the ultrastructure. Third, the CD spectrum of the unmodified hCT minimal recognition module under the same conditions and at similar concentrations showed an α-helical structure.

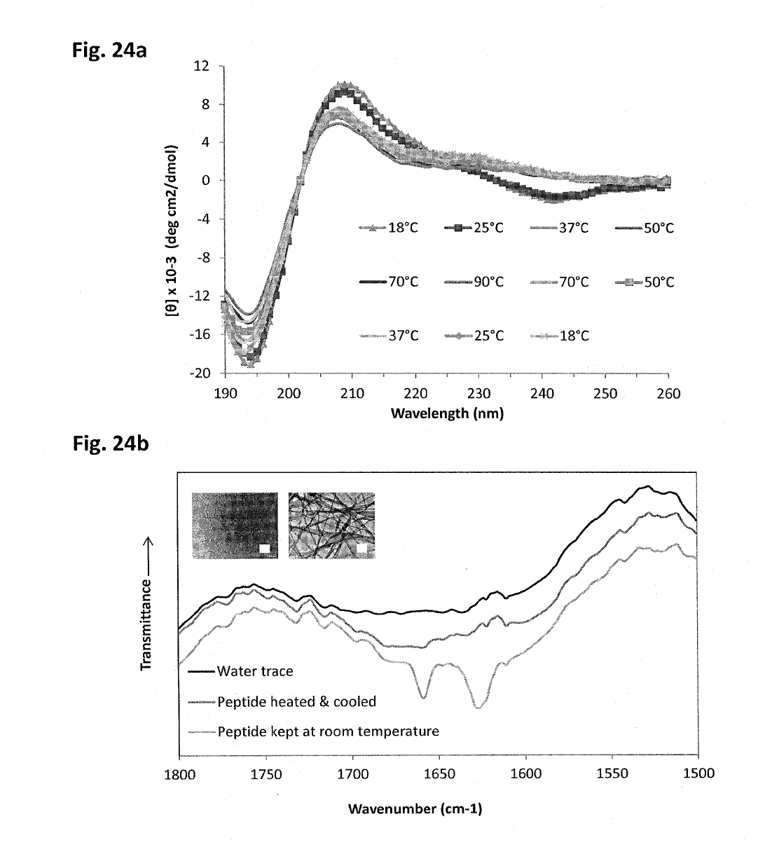

To further elucidate the structural properties of Asp-DOPA-Asn-Lys-DOPA, temperature-dependent CD was performed. Freshly made 6 mM peptide solutions in water were diluted to a final concentration of 0.15 mM and CD spectra were collected during a stepwise increase in temperature from 18° C. to 90° C. and a subsequent stepwise decrease to 18° C. Throughout this process, the spectral profile retained its distinct features (FIG. 24A). However, intensity loss was observed as the temperature increased, with a significant loss occurring near the 209 nm band and in the 235-250 nm region. This effect seemed irreversible as only little intensity gain was observed upon temperature decrease. Subsequent FTIR analysis of the CD cuvette content showed no bands in the amide I′ region while a control sample, taken from the same solution and kept at room temperature, displayed the characteristic Asp-DOPA-Asn-Lys-DOPA FTIR spectrum with peak positions at 1627 and 1659 cm-1 (FIG. 24B). Moreover, in a TEM examination, the solution that was subjected to temperature variations did not contain assemblies (FIG. 24B1—insert) while the control solution contained fibrillar assemblies (FIG. 24B2—insert). Taken together, these results suggest that the ultrastructure is impaired by elevated temperatures in a process accompanied by aggregation and sedimentation of the peptide.

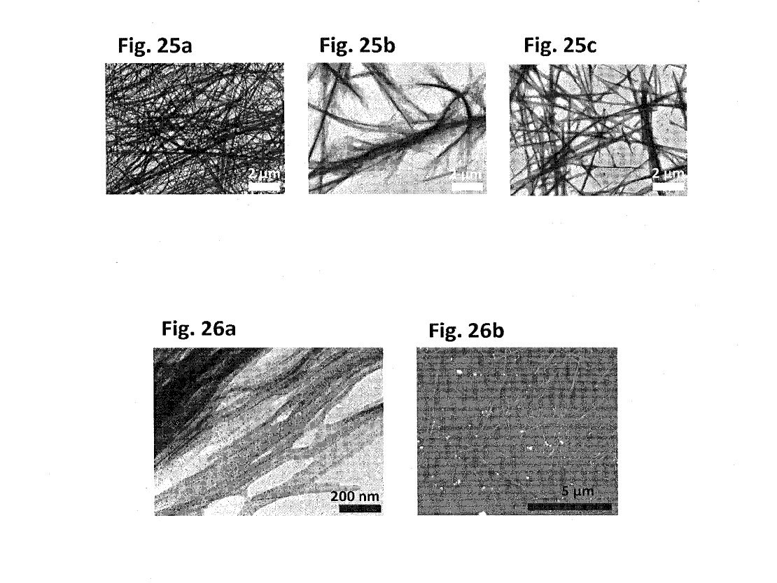

Since the first significant change in the CD signal was observed when the temperature was increased from 25° C. to 37° C., we sought to examine the ultrastructural effect of subjecting the assemblies to this particular temperature. To this end, a peptide solution at a concentration of 6 mM was allowed to self-assemble at room temperature for four days then incubated overnight at 37° C. TEM samples were taken from this solution immediately after incubation as well as after 8 h of recovery at room temperature. A third sample was taken from a solution aliquot incubated at room temperature as control. While the aliquot incubated at room temperature contained a dense network of fibrillar assemblies with characteristic morphology (FIG. 25A), the aliquot incubated at 37° C. contained fewer assemblies, from which fine fibrillar protrusions were extending (FIG. 25B). This morphological transition correlates with the CD signal intensity loss upon heating to 37° C. Moreover, in accordance with the CD results, this transition does not seem to be reversible as the characteristic morphology was not fully retained following recovery in room temperature (FIG. 25C). Since the morphological transition was accompanied by a decrease in the abundance of assemblies, it follows that this ultrastructural reorganization process leads to the aggregation and sedimentation of the assemblies.

### Functional Examination

The ability of Asp-DOPA-Asn-Lys-DOPA assemblies to reduce ionic silver was examined. An aqueous peptide solution was produced by means of repeated pelleting in water to remove any peptide monomers. Subsequently, AgNO3 solution was used for resuspention of the pellet and the solution was incubated for 15 min then re-pelleted and resuspended in water. TEM examination of the resultant solution revealed significant deposition of silver on the fibrillar assemblies which appeared as dark nanometric clusters (FIG. 26A), while this was not observed in a control solution to which AgNO3 was not added. Furthermore, the clusters seemed to selectively deposit on the assemblies compared to the background. Similar results were obtained in an E-SEM examination, with the clusters appearing in white (FIG. 26B). The results show that Asp-DOPA-Asn-Lys-DOPA assemblies possess the ability to reduce ionic silver while retaining their ultrastructure in solution.

In conclusion, as demonstrated herein, a DOPA-incorporated pentapeptide inspired by a minimal amyloid recognition module can self-assemble into an amyloid-like supramolecular polymer of fibrillar nature in simple aqueous solutions. The assemblies formed were investigated by electron microscopy, amyloidophilic dyes and spectroscopic methods. The investigation revealed that the supramolecular polymer formed is endowed with characteristics of both amyloids and DOPA-containing polypeptides. Furthermore, the ability to reduce ionic silver while maintaining the ultrastructural integrity has demonstrated the applicative potential of this novel nanobiomaterial.

While the present invention has been particularly described, persons skilled in the art will appreciate that many variations and modifications can be made. Therefore, the invention is not to be construed as restricted to the particularly described embodiments, and the scope and concept of the invention will be more readily understood by reference to the claims, which follow.

### REFERENCES

1. Lloyd D. Graham et al., Biomacromolecules 2005, 6, 3300-3312.
2. Waite, J., Adhesion à la Moule. Integrative and Comparative Biology, 2002. 42: p. 1172-1180.
3. Waite, J., et al., Mussel Adhesion: Finding the Tricks Worth Mimicking. The Journal of Adhesion, 2005. 81(3-4): p. 297-317.
4. Wiegemann, M., Adhesion in blue mussels (Mytilus edulis) and barnacles (genus Balanus): Mechanisms and technical applications. Aquatic Sciences, 2005. 67(2): p. 166-176.
5. Catron, N. D., H. Lee, and P. B. Messersmith, Enhancement of poly(ethylene glycol) mucoadsorption by biomimetic end group functionalization. Biointerphases, 2006. 1(4): p. 134-141.
6. Dalsin, J. L., et al., Versatile mussel adhesive-inspired biomimetic antifouling polymers. Abstracts of Papers of the American Chemical Society, 2006. 231.
7. Doraiswamy, A., et al., Matrix-assisted pulsed-laser evaporation of DOPA-modified poly(ethylene glycol) thin films. Journal of Adhesion Science and Technology, 2007. 21(3-4): p. 287-299.
8. Huang, K., et al., Synthesis and characterization of self-assembling block copolymers containing bioadhesive end groups. Biomacromolecules, 2002. 3(2): p. 397-406.
9. Statz, A. R., et al., New peptidomimetic polymers for antifouling surfaces. Journal of the American Chemical Society, 2005. 127(22): p. 7972-7973.
10. Gazit, E., Self-assembled peptide nanostructures: the design of molecular building blocks and their technological utilization. Chemical Society Reviews, 2007. 36(8): p. 1263-1269.
11. Azriel, R. and E. Gazit, Analysis of the minimal amyloid-forming fragment of the islet amyloid polypeptide. An experimental support for the key role of the phenylalanine residue in amyloid formation. J Biol Chem, 2001. 276(36): p. 34156-61.
12. Gazit, E., A possible role for pi-stacking in the self-assembly of amyloid fibrils. FASEB J, 2002. 16(1): p. 77-83.
13. Mazor, Y., et al., Identification and characterization of a novel molecular-recognition and self-assembly domain within the islet amyloid polypeptide. J Mol Biol, 2002. 322(5): p. 1013-24.
14. Reches, M. and E. Gazit, Casting metal nanowires within discrete self-assembled peptide nanotubes. Science, 2003. 300(5619): p. 625-7.
15. Reches, M., Y. Porat, and E. Gazit, Amyloid fibril formation by pentapeptide and tetrapeptide fragments of human calcitonin. J Biol Chem, 2002. 277(38): p. 35475-80.
16. Reches, M. and E. Gazit, Controlled patterning of aligned self-assembled peptide nanotubes. Nat Nanotechnol, 2006. 1(3): p. 195-200.
17. Adler-Abramovich, L., et al., Self-assembled arrays of peptide nanotubes by vapour deposition. Nat Nanotechnol, 2009. 4(12): p. 849-54.
18. Silverman, H. G. and F. F. Roberto, Understanding Marine Mussel Adhesion. Marine Biotechnology, 2007. 9(6): p. 661-681.
19. Balbach, J. J. I., Y.; Antzutkin, O. N.; Leapman, R. D.; and N. W. D. Rizzo, F.; Reed, J.; Tycko, R. B, Amyloid Fibril Formation by Aβ 16-22, a Seven-Residue Fragment of the Alzheimer's Aβ-Amyloid Peptide, and Structural Characterization by Solid State NMR. Biochemistry, 2000. 39: p. 13748-13759.
20. Lu, K., et al., Exploiting amyloid fibril lamination for nanotube self-assembly. J Am Chem Soc, 2003. 125(21): p. 6391-3.
21. Black, K. C., Z. Liu, and P. B. Messersmith, Catechol Redox Induced Formation of Metal Core-Polymer Shell Nanoparticles. Chem Mater, 2011. 23(5): p. 1130-1135.
22. Fullenkamp, D. E., et al., Mussel-inspired silver-releasing antibacterial hydrogels. Biomaterials, 2012. 33(15): p. 3783-91.
23. Saroj Kumar Roy and Bala Prabhakar, Bioadhesive Polymeric Platforms for Transmucosal Drug Delivery Systems—a Review, Tropical Journal of Pharmaceutical Research, February 2010; 9 (1): p. 91-104.
24. a) J. Wang, C. S. Liu, X. Lu, M. Yin, Biomaterials 2007, 28, 3456; b) M. Yin, Y. Yuan, C. S. Liu, J. Wang, Biomaterials 2009, 30, 2764.
25. J. Sedo, J. Saiz-Poseu, F. Busque, D. Ruiz-Molina, Adv. Mater. 2013, 25, 653.
26. E. Faure, C. Falentin-Daudre, C. Jerome, J. Lyskawa, D. Fournier, P. Woisel, C. Detrembleur, Prog. Polym. Sci. 2013, 38, 236.
27. a) K. C. L. Black, Z. Q. Liu, P. B. Messersmith, Chem. Mater. 2011, 23, 1130; b) D. E. Fullenkamp, J. G. Rivera, Y. K. Gong, K. H. A. Lau, L. H. He, R. Varshney, P. B. Messersmith, Biomaterials 2012, 33, 3783.
28. R. Orbach, I. Mironi-Harpaz, L. Adler-Abramovich, E. Mossou, E. P. Mitchell, V. T. Forsyth, E. Gazit, D. Seliktar, Langmuir 2012, 28, 2015.

What is claimed is:

1-41. (canceled)

42. A self-assembled micro- or nano-structure comprising (i) a plurality of aromatic amino acids selected from 3,4-dihydroxyphenyl-L-alanine (DOPA) and a DOPA-derivative; or (ii) a plurality of peptides, each peptide comprising between 2 and 9 amino acids, at least one of which is an aromatic amino acid selected from 3,4-dihydroxyphenyl-L-alanine (DOPA) and a DOPA-derivative; or (iii) a combination of said amino acids and peptides; wherein said micro- or nano-structure has at least one property selected from bioadhesive, anti-oxidant, anti-fouling, anti-bacterial and any combination thereof.

43. The micro- or nano-structure of claim 42, which is selected from the group consisting of a fibrillar micro- or nano-structure, a tubular micro- or nano-structure, a spherical micro- or nano-structure and a ribbon-like micro- or nano-structure.

44. The micro- or nano-structure of claim 43, which is at least about 1 nm in diameter, and which does not exceed about 500 nm in diameter.

45. The micro- or nano-structure of claim 42, wherein each peptide in said plurality of peptides comprises between 2 and 7 amino acids.

46. The micro- or nano-structure of claim 42, comprising a combination of said amino acids and said peptides.

47. The micro- or nano-structure of claim 42, wherein each peptide in said plurality of peptides comprises a plurality of aromatic amino acids selected from 3,4-dihydroxyphenyl-L-alanine (DOPA), a DOPA-derivative and a combination thereof.

48. The micro- or nano-structure of claim 42, wherein at least one peptide in said plurality of peptides is a 3,4-dihydroxyphenyl-L-alanine-(3,4-dihydroxyphenyl-L-alanine) (DOPA-DOPA) homodipeptide.

49. The micro- or nano-structure of claim 42, wherein at least one peptide in said plurality of peptides incorporates at least one 3,4-dihydroxyphenyl-L-alanine-(3,4-dihydroxyphenyl-L-alanine) (DOPA-DOPA) homodipeptide in the peptide backbone.

50. The micro- or nano-structure of claim 48, wherein said DOPA-DOPA homopeptide further comprises at least one end-capping modified moiety at the C- or N-terminus.

51. The micro- or nano-structure of claim 42, wherein at least one amino acid or peptide in said plurality of amino acids or peptides further comprises at least one amino acid capable of enhancing cohesion, enhancing adhesion of said peptide to a surface, or a combination thereof.

52. The micro- or nano-structure of claim 51, wherein said amino acid is charged at neutral pH, wherein said amino acid comprises a positively charged side chain capable of ionically interacting with negatively charged surface, or a negatively charged side chain capable of ionically interacting with positively charged surface.

53. The micro- or nano-structure of claim 52, wherein said amino acid is selected from the group consisting of lysine, ornithine, arginine, aspartic acid, glutamic acid, and histidine.

54. The micro- or nano-structure of claim 42, wherein at least one amino acid or peptide in said plurality of amino acids or peptides comprises at least one end-capping modified moiety at the C- or N-terminus.

55. The micro- or nano-structure of claim 54, wherein said end capping moiety is selected from the group consisting of an aromatic end capping moiety and a non-aromatic end-capping moiety.

56. The micro- or nano-structure of claim 55, wherein said aromatic end capping moiety is selected from the group consisting of 9-fluorenylmethyloxycarbonyl (Fmoc), benzyloxycarbonyl (Cbz), naphthalene (Nap) derivatives, phenothiazine (PTZ), azobenzene (Azo), pyrene (Pyr), and cinnamoyl.

57. The micro- or nano-structure of claim 55, wherein said non-aromatic end capping moiety is selected from the group consisting of acetyl and tert-butoxycarbonyl (Boc).

58. The micro- or nano-structure of claim 54, wherein said end-capping moiety comprises a labeling moiety.

59. The micro- or nano-structure of claim 42, wherein at least one of the plurality of amino acids or peptides is selected from the group consisting of Fmoc-DOPA, DOPA-DOPA, DOPA-Phe-Phe, Fmoc-DOPA-DOPA, Fmoc-DOPA-DOPA-Lys, Fmoc-Phe-Phe-DOPA-DOPA-Lys, Lys-Leu-Val-DOPA-DOPA-Ala-Glu, Asp-DOPA-Asn-Lys-DOPA and derivatives of any of the foregoing comprising an end capping moiety, preferably an Fmoc moiety.

60. The micro- or nano-structure of claim 42, which comprises:
(i) a plurality of aromatic amino acids selected from 3,4-dihydroxyphenyl-L-alanine (DOPA), a DOPA-derivative and combinations thereof; or a plurality of peptides comprising at least one aromatic amino acids selected from 3,4-dihydroxyphenyl-L-alanine (DOPA), a DOPA-derivative and combinations thereof;
(ii) at least one amino acid which is charged at neutral pH; and
(iii) optionally, at least one additional amino acid selected from the group consisting of naturally occurring amino acids, synthetic amino acids and combinations thereof, wherein said micro- or nano-structure is bio-adhesive.

61. The micro- or nano-structure of claim 42, which is provided in the form of a hydrogel.

62. The micro- or nano-structure of claim 42, which is co-assembled with one or more additional self-assembled peptides, polypeptides, polysaccharides, polymers, or a combination thereof.

63. The micro- or nano-structure of claim 62, wherein the additional self-assembled peptide is selected from Phe-Phe and Fmoc Phe-Phe, wherein Phe is phenylalanine.

64. A method of generating the self-assembled micro- or nano-structure of claim 1, the method comprising incubating a plurality of amino acids or peptides under conditions which favor formation of said micro- and nano-structure.

65. The micro- or nano-structure of claim 42, for use in preparation of pharmaceutical composition, cosmetic composition, a medical device or a medical device coating.

66. The micro- or nano-structure of claim 65, for use in preparation of biological glue.

67. The micro- or nano-structure of claim 65, for use as an anti-oxidant, a radical trapper, a metal chelator, or an oxidizable reducing agent.

68. A method of combating bacteria, comprising the step of contacting the bacteria with the micro- or nano-structure of claim 42.

69. A method of disinfecting a surface, comprising the step of contacting said surface with the micro- or nano-structure of claim 42.

70. A pharmaceutical composition, cosmetic composition, a medical device or a medical device coating, comprising the self-assembled micro- or nano-structure of claim 42, and a pharmaceutically or cosmetically acceptable carrier.

71. A kit for forming the self-assembled micro- or nano-structure of claim 42, the kit comprising (i) a plurality of aromatic amino acids selected from 3,4-dihydroxyphenyl-L-alanine (DOPA) and a DOPA-derivative; or a plurality of peptides, each peptide comprising between 2 and 9 amino acids, at least one of which is an aromatic amino acid selected from 3,4-dihydroxyphenyl-L-alanine (DOPA) and a DOPA-derivative; or a combination of said amino acids and peptides and (ii) an aqueous solution, each being individually packaged within the kit,
wherein said plurality of amino acids or peptides and said solution are selected such that upon contacting said plurality of peptides and said solution, said micro- or nano-structure is formed.

72. A composition comprising the micro- or nano-structure of claim 42, and an agent selected from the group consisting of a therapeutically active agent, a diagnostic agent, a biological substance and a labeling moiety; wherein the micro- or nano-structure optionally encapsulates said agent, or is attached to said agent.

73. The composition according to claim 72, wherein the agent is selected from the group consisting of drugs, cells, proteins, enzymes, hormones, growth factors, nucleic acids, organisms such as bacteria, fluorescence compounds or moieties, phosphorescence compounds or moieties, and radioactive compounds or moieties.


SEQUENCE LISTING (2 sequences)

SEQ ID NO: 1 - protein, 32 aa, Homo sapiens
CGNLSTCMLGTYTQDFNKFHTFPQTAIGVGAP

SEQ ID NO: 2 - protein, 5 aa, Artificial Sequence
  Synthetic peptide
DFNKF